SEQUENCE LISTING (45 sequences)

SEQ ID NO: 1 - protein, 124 aa, synthetic construct
  REGION 1..124: note = Synthetic
QVQLVESGGGVVQPGRSLRLSCAASGFTFSSYGMHWVRQAPGKGLEWVAFMSYDGSNKFYSDSVKGRFTISRDNSRKMLFLQMNNLRAEDTAVYYCARDQYYDFLTDHGVFDYWGQGTLVTVSS

SEQ ID NO: 2 - protein, 8 aa, synthetic construct
  REGION 1..8: note = Synthetic
GFTFSSYG

SEQ ID NO: 3 - protein, 8 aa, synthetic construct
  REGION 1..8: note = Synthetic
MSYDGSNK

SEQ ID NO: 4 - protein, 17 aa, synthetic construct
  REGION 1..17: note = Synthetic
ARDQYYDFLTDHGVFDY

SEQ ID NO: 5 - protein, 122 aa, synthetic construct
  REGION 1..122: note = Synthetic
QVQLQESGPGLVKPSETLSLTCTVSGGSISSYYWSWIRQPPGKGLEWIGYIYYSGITHYNPSLKSRVTISVDTSKIQFSLKLSSVTAADTAVYYCARWGVRRDYYYYGMDVWGQGTTVTVSS

SEQ ID NO: 6 - protein, 8 aa, synthetic construct
  REGION 1..8: note = Synthetic
GGSISSYY

SEQ ID NO: 7 - protein, 7 aa, synthetic construct
  REGION 1..7: note = Synthetic
IYYSGIT

SEQ ID NO: 8 - protein, 16 aa, synthetic construct
  REGION 1..16: note = Synthetic
ARWGVRRDYYYYGMDV

SEQ ID NO: 9 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 10 - protein, 12 aa, synthetic construct
  REGION 1..12: note = Synthetic
RASQSVSSSYLA

SEQ ID NO: 11 - protein, 7 aa, synthetic construct
  REGION 1..7: note = Synthetic
GASSRAT

SEQ ID NO: 12 - protein, 9 aa, synthetic construct
  REGION 1..9: note = Synthetic
QQYGSSPWT

SEQ ID NO: 13 - protein, 450 aa, synthetic construct
  REGION 1..450: note = Synthetic
QVQLVESGGGVVQPGRSLRLSCAASGFTFSSYGMHWVRQAPGKGLEWVAFMSYDGSNKFYSDSVKGRFTISRDNSRKMLFLQMNNLRAEDTAVYYCARDQYYDFLTDHGVFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 448 aa, synthetic construct
  REGION 1..448: note = Synthetic
QVQLQESGPGLVKPSETLSLTCTVSGGSISSYYWSWIRQPPGKGLEWIGYIYYSGITHYNPSLKSRVTISVDTSKIQFSLKLSSVTAADTAVYYCARWGVRRDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 15 - protein, 215 aa, synthetic construct
  REGION 1..215: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 124 aa, synthetic construct
  REGION 1..124: note = Synthetic
QVQLVESGGGVVQPGRSLRLSCAASGFTFSSYGMHWVRQAPGKGLEWVAVISYAGNNKYYADSVKGRFTVSRDNSKKTLYLQMNSLRSEDTAVYYCAKDSYYDFLTDPDVLDIWGQGTMVTVSS

SEQ ID NO: 17 - protein, 8 aa, synthetic construct
  REGION 1..8: note = Synthetic
GFTFSSYG

SEQ ID NO: 18 - protein, 8 aa, synthetic construct
  REGION 1..8: note = Synthetic
ISYAGNNK

SEQ ID NO: 19 - protein, 17 aa, synthetic construct
  REGION 1..17: note = Synthetic
AKDSYYDFLTDPDVLDI

SEQ ID NO: 20 - protein, 129 aa, synthetic construct
  REGION 1..129: note = Synthetic
EVQLVESGGGLVQPGGSLRLSCAASGFTFSTYAMTWVRQAPGKGLEWVSFISGSGDNTYYADSVKGRFTISRDNSKNTLYLQMNSLRADDSAIYHCAKDSGYCSSGGCSLYFYYGMDVWGQGTTVTVSS

SEQ ID NO: 21 - protein, 8 aa, synthetic construct
  REGION 1..8: note = Synthetic
GFTFSTYA

SEQ ID NO: 22 - protein, 8 aa, synthetic construct
  REGION 1..8: note = Synthetic
ISGSGDNT

SEQ ID NO: 23 - protein, 22 aa, synthetic construct
  REGION 1..22: note = Synthetic
AKDSGYCSSGGCSLYFYYGMDV

SEQ ID NO: 24 - protein, 118 aa, synthetic construct
  REGION 1..118: note = Synthetic
EVQLVESGGGLVQPGGSLRLSCAASGFTFSRNNMHWVRQAPGKGLEYVSGISSNGGRTYYADSVKGRFTISRDNSKNTLYLQMGGLRAADMAVYFCTRDDELLSFDYWGQGTLVTVSS

SEQ ID NO: 25 - protein, 8 aa, synthetic construct
  REGION 1..8: note = Synthetic
GFTFSRNN

SEQ ID NO: 26 - protein, 8 aa, synthetic construct
  REGION 1..8: note = Synthetic
ISSNGGRT

SEQ ID NO: 27 - protein, 11 aa, synthetic construct
  REGION 1..11: note = Synthetic
TRDDELLSFDY

SEQ ID NO: 28 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 29 - protein, 6 aa, synthetic construct
  REGION 1..6: note = Synthetic
QSISSY

SEQ ID NO: 30 - skipped

SEQ ID NO: 31 - protein, 10 aa, synthetic construct
  REGION 1..10: note = Synthetic
QQSYSTPPIT

SEQ ID NO: 32 - protein, 450 aa, synthetic construct
  REGION 1..450: note = Synthetic
QVQLVESGGGVVQPGRSLRLSCAASGFTFSSYGMHWVRQAPGKGLEWVAVISYAGNNKYYADSVKGRFTVSRDNSKKTLYLQMNSLRSEDTAVYYCAKDSYYDFLTDPDVLDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 455 aa, synthetic construct
  REGION 1..455: note = Synthetic
EVQLVESGGGLVQPGGSLRLSCAASGFTFSTYAMTWVRQAPGKGLEWVSFISGSGDNTYYADSVKGRFTISRDNSKNTLYLQMNSLRADDSAIYHCAKDSGYCSSGGCSLYFYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 34 - protein, 444 aa, synthetic construct
  REGION 1..444: note = Synthetic
EVQLVESGGGLVQPGGSLRLSCAASGFTFSRNNMHWVRQAPGKGLEYVSGISSNGGRTYYADSVKGRFTISRDNSKNTLYLQMGGLRAADMAVYFCTRDDELLSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 35 - protein, 215 aa, synthetic construct
  REGION 1..215: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 117 aa, synthetic construct [sequence omitted]

SEQ ID NO: 37 - protein, 8 aa, synthetic construct
  REGION 1..8: note = Synthetic
GFTFSNFG

SEQ ID NO: 38 - protein, 8 aa, synthetic construct
  REGION 1..8: note = Synthetic
ISGGGRDT

SEQ ID NO: 39 - protein, 10 aa, synthetic construct
  REGION 1..10: note = Synthetic
VKWGNIYFDY

SEQ ID NO: 40 - protein, 107 aa, synthetic construct [sequence omitted]

SEQ ID NO: 41 - protein, 6 aa, synthetic construct
  REGION 1..6: note = Synthetic
LSINTF

SEQ ID NO: 42 - skipped

SEQ ID NO: 43 - protein, 9 aa, synthetic construct
  REGION 1..9: note = Synthetic
QQSSNTPFT

SEQ ID NO: 44 - protein, 444 aa, synthetic construct
  REGION 1..444: note = Synthetic
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 214 aa, synthetic construct
  REGION 1..214: note = Synthetic
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0043560 A1
(43) Pub. Date: Feb. 8, 2024

(54) Methods of Treating Metastatic Castration-Resistant Prostate Cancer with Bispecific Anti-PSMA x Anti-CD28 Antibodies in Combination with Anti-PD-1 Antibodies

(71) Applicant: Regeneron Pharmaceuticals, Inc., Tarrytown, NY (US)

(72) Inventors: Elizabeth Miller, Pelham, NY (US); Sabina Sandigursky, Long Island City, NY (US); Israel Lowy, Dobbs Ferry, NY (US); Frank Seebach, Montclair, NJ (US)

(21) Appl. No.: 18/229,117

(22) Filed: Aug. 1, 2023

Related U.S. Application Data
(60) Provisional application No. 63/394,492, filed on Aug. 2, 2022.
(60) Provisional application No. 63/420,186, filed on Oct. 28, 2022.
(60) Provisional application No. 63/463,655, filed on May 3, 2023.

(51) Int. Cl. C07K 16/30; C07K 16/28; A61P 35/04

(52) U.S. Cl. CPC C07K 16/3069; C07K 16/2818; C07K 16/2866; A61P 35/04; A61K 2039/507

(57) ABSTRACT

The present disclosure provides methods for treating, reducing the severity, or inhibiting the growth of cancer (e.g., prostate cancer or metastatic castration-resistant prostate cancer). The methods of the present disclosure comprise administering to a subject in need thereof a therapeutically effective amount of a bispecific antibody or antigen-binding fragment thereof that specifically binds prostate-specific membrane antigen (PSMA) and CD28 in combination with an antibody or antigen-binding fragment thereof that specifically binds to programmed death receptor-1 (PD-1).

### CROSS REFERENCE TO RELATED APPLICATIONS

This application claims the benefit under 35 USC § 119(e) of U.S. Provisional Application Nos. 63/394,492, filed Aug. 2, 2022; 63/420,186, filed Oct. 28, 2022; and 63/463,655, filed May 3, 2023, each of which is incorporated herein by reference in its entirety for all purposes.

### REFERENCE TO A SEQUENCE LISTING

This application incorporates by reference a computer readable Sequence Listing in ST.26 XML format, titled 11051US01_Sequence, created on Jul. 28, 2023 and containing 58,900 bytes.

### FIELD OF THE INVENTION

The present invention relates to methods for treating cancer comprising administering to a subject in need thereof a therapeutically effective amount of a bispecific antibody that specifically binds to prostate-specific membrane antigen (PSMA) and CD28 in combination with an antibody that specifically binds to programmed death receptor-1 (PD-1).

### BACKGROUND

Prostate-specific membrane antigen (PSMA), also known as FOLH1, glutamate carboxypeptidase II (GCPII), N-acetyl-L-aspartyl-L-glutamate peptidase I (NAALADase I), or N-acetyl-aspartylglutamate (NAAG) peptidase, is a homodimeric, enzymatic type II transmembrane protein encoded by the folate hydrolase 1 (FOLH1) gene. PSMA is an integral, non-shed membrane glycoprotein highly expressed on malignant prostate tissue and is a cell-surface marker for prostate cancer, but shows limited expression on normal tissue. Its expression is maintained in castrate-resistant prostate cancer, a condition with poor outcome and limited treatment options. Methods for treating prostate cancer by targeting PSMA have been investigated. For example, Yttrium-90 capromab is a radiotherapeutic comprising a monoclonal antibody to an intracellular epitope of PSMA. In another example, J591, a monoclonal antibody to an extracellular epitope of PSMA, is part of the radiotherapeutic Lutetium-177 J591 and in MLN2704, in which maytansinoid 1 (DM1, an antimicrotubule agent) is conjugated to J591. These therapies have been associated with toxicity. PSMA is also expressed within the neovasculature of other tumors such as bladder, renal, gastric, and colorectal carcinomas.

CD28 is a type I transmembrane protein, which has a single extracellular Ig-V-like domain assembled as a homodimer and which is expressed on the surface of T cells. CD28 is the receptor for the CD80 (B7.1) and CD86 (B7.2) proteins and is activated by CD80 or CD86 expressed on antigen-presenting cells (APCs). The binding of CD28 to CD80 or CD86 provides co-stimulatory signals important for T cell activation and survival. T cell stimulation through CD28, in addition to the T-cell receptor (TCR), provides a potent signal for the production of various interleukins. CD28 also potentiates cellular signals such as pathways controlled by the NFκB transcription factor after TCR activation. The CD28 co-signal is important for effective T-cell activation such as T cell differentiation, proliferation, cytokine release and cell-death. Anti-CD28 antibodies have been proposed for therapeutic purposes involving the activation of T cells. One particular anti-CD28 antibody, TGN1412 (anti-CD28 superagonist), was used in a clinical trial in 2006, in which six healthy volunteers were dosed intravenously with TGN1412 (anti-CD28 superagonist) at a dose of 0.1 mg/kg. Within two hours, all six patients had significant inflammatory responses (cytokine storm), and all patients were in multi-organ failure within sixteen hours. Subjects were treated with corticosteroids, and cytokine levels returned to normal within 2-3 days (Suntharalingam, et al., Cytokine Storm in a Phase 1 Trial of the Anti-CD28 Monoclonal Antibody TGN1412, NEJM 355:1018-1028 (2006)).

Programmed death receptor-1 (PD-1) signaling in the tumor microenvironment plays a key role in allowing tumor cells to escape immune surveillance by the host immune system. Blockade of the PD-1 signaling pathway has demonstrated clinical activity in patients with multiple tumor types, and antibody therapeutics that block PD-1 (e.g., nivolumab and pembrolizumab) have been approved for the treatment of metastatic melanoma and metastatic squamous non-small cell lung cancer. Recent data has demonstrated the clinical activity of PD-1 blockade in patients with aggressive NHL and Hodgkin's lymphoma (Lesokhin, et al. 2014, Abstract 291, 56th ASH Annual Meeting and Exposition, San Francisco, Calif.; Ansell et al. 2015, N. Engl. J. Med. 372(4):311-9).

Prostate cancer is the leading cause of new cancer diagnoses and the second most common cause of cancer-related death in men in the United States. There were 1.3 million new cases of prostate cancer and 358,989 deaths estimated worldwide in 2018. Therapies blocking androgen related pathways have been the standard for decades in treating prostate cancers. However, patients progress on androgen depletion and/or surgical castration and develop castration resistant prostate cancer. Prognosis is especially poor for men with metastatic castration resistant prostate cancer (mCRPC). Currently, metastatic prostate cancers remain incurable and improvement in long-term survival remains a high unmet need.

### SUMMARY

According to certain embodiments, the present disclosure provides methods for treating, ameliorating at least one symptom or indication, or inhibiting the growth of a PSMA-expressing cancer in a subject. The methods according to this aspect of the disclosure comprise administering a therapeutically effective amount of a bispecific antibody or antigen-binding fragment thereof that specifically binds to prostate specific membrane antigen (PSMA) and CD28 in combination with an antibody or antigen-binding fragment thereof that specifically binds to programmed death receptor-1 (PD-1) to a subject in need thereof.

In certain embodiments of the present disclosure, methods are provided for treating, ameliorating at least one symptom or indication, or inhibiting the growth of a PSMA-expressing cancer in a subject. In certain embodiments of the present disclosure, methods are provided for delaying the growth of a tumor or preventing tumor recurrence. The methods, according to this and other aspects of the disclosure, comprise sequentially administering one or more doses of a therapeutically effective amount of a bispecific anti-PSMA x anti-CD28 antibody or antigen-binding fragment thereof in combination with one or more doses of a therapeutically effective amount of an anti-PD-1 antibody or antigen-binding fragment thereof to a subject in need thereof.

In one aspect, the present disclosure provides a method of treating a PSMA-expressing cancer in a subject in need thereof, comprising administering to the subject a combination of a bispecific antibody or antigen-binding fragment thereof comprising a first antigen-binding domain that specifically binds prostate specific membrane antigen (PSMA) on a target tumor cell, and a second antigen-binding domain that specifically binds human CD28 on a T cell, and an antibody or antigen-binding fragment thereof that specifically binds programmed death receptor-1 (PD-1), wherein the bispecific antibody is administered to the subject at a dose of at least 0.03 mg.

In some embodiments, the PSMA-expressing cancer is prostate cancer. In some cases, the PSMA-expressing cancer is metastatic prostate cancer. In some cases, the PSMA-expressing cancer is castration-resistant prostate cancer.

In some embodiments, the subject has received at least two prior therapies for metastatic and/or castration-resistant prostate cancer. In some cases, the subject has received at least one anti-androgen therapy. In some embodiments, the anti-androgen therapy is selected from abiraterone, enzalutamide, apalutamide, or darolutamide.

In some embodiments, the subject has histologically or cytologically confirmed adenocarcinoma of the prostate without pure small cell carcinoma.

In some embodiments, the subject has metastatic castration-resistant prostate cancer with a prostate specific antigen (PSA) value of ≥4 ng/ml prior to treatment with the bispecific antibody. In some cases, the subject's cancer has progressed within a six month period prior to treatment with the bispecific antibody, wherein cancer progression is determined by: (a) a rising PSA level confirmed with an interval of 1 week between each assessment; (b) radiographic disease progression in soft tissue with or without a rise in PSA; and/or (c) radiographic disease progression in bone with an appearance of two or more bone lesions on bone scan with or without a rise in PSA.

In some embodiments, the subject has had an orchiectomy. In some embodiments, the subject is receiving luteinizing hormone-releasing hormone (LHRH) agonist or antagonist therapy, and has a serum testosterone level of <50 ng/ml prior to treatment with the bispecific antibody.

In some embodiments, the first antigen-binding domain of the bispecific antibody comprises: (a) three heavy chain complementarity determining regions (HCDR1, HCDR2 and HCDR3) contained within a heavy chain variable region (HCVR) comprising the amino acid sequence of SEQ ID NO: 1; and (b) three light chain complementarity determining regions (LCDR1, LCDR2 and LCDR3) contained within a light chain variable region (LCVR) comprising the amino acid sequence of SEQ ID NO: 9. In some cases, the first antigen-binding domain comprises a HCDR1 comprising the amino acid sequence of SEQ ID NO: 2, a HCDR2 comprising the amino acid sequence of SEQ ID NO: 3, and a HCDR3 comprising the amino acid sequence of SEQ ID NO: 4. In some cases, the first antigen-binding domain comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12. In some embodiments, the first antigen-binding domain comprises a HCVR comprising the amino acid sequence of SEQ ID NO: 1, and a LCVR comprising the amino acid sequence of SEQ ID NO: 9.

In some embodiments, the second antigen-binding domain of the bispecific antibody comprises: (a) three heavy chain complementarity determining regions (HCDR1, HCDR2 and HCDR3) contained within a heavy chain variable region (HCVR) comprising the amino acid sequence of SEQ ID NO: 5; and (b) three light chain complementarity determining regions (LCDR1, LCDR2 and LCDR3) contained within a light chain variable region (LCVR) comprising the amino acid sequence of SEQ ID NO: 9. In some cases, the second antigen-binding domain comprises a HCDR1 comprising the amino acid sequence of SEQ ID NO: 6, a HCDR2 comprising the amino acid sequence of SEQ ID NO: 7, and a HCDR3 comprising the amino acid sequence of SEQ ID NO: 8. In some cases, the second antigen-binding domain comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12. In some embodiments, the second antigen-binding domain comprises a HCVR comprising the amino acid sequence of SEQ ID NO: 5, and a LCVR comprising the amino acid sequence of SEQ ID NO: 9.

In some embodiments, the bispecific antibody comprises a first heavy chain comprising the amino acid sequence of SEQ ID NO: 13.

In some embodiments, the bispecific antibody comprises a second heavy chain comprising the amino acid sequence of SEQ ID NO: 14.

In some embodiments, the bispecific antibody comprises a first heavy chain comprising the amino acid sequence of SEQ ID NO: 13, a second heavy chain comprising the amino acid sequence of SEQ ID NO: 14, and a common light chain comprising the amino acid sequence of SEQ ID NO: 15.

In some embodiments, the first antigen-binding domain of the bispecific antibody comprises: (a) three heavy chain complementarity determining regions (HCDR1, HCDR2 and HCDR3) contained within a heavy chain variable region (HCVR) comprising the amino acid sequence of SEQ ID NO: 16; and (b) three light chain complementarity determining regions (LCDR1, LCDR2 and LCDR3) contained within a light chain variable region (LCVR) comprising the amino acid sequence of SEQ ID NO: 28. In some cases, the first antigen-binding domain comprises a HCDR1 comprising the amino acid sequence of SEQ ID NO: 17, a HCDR2 comprising the amino acid sequence of SEQ ID NO: 18, and a HCDR3 comprising the amino acid sequence of SEQ ID NO: 19. In some cases, the first antigen-binding domain comprises a LCDR1 comprising the amino acid sequence of SEQ ID NO: 29, a LCDR2 comprising the amino acid sequence of SEQ ID NO: 30, and a LCDR3 comprising the amino acid sequence of SEQ ID NO: 31. In some cases, the first antigen-binding domain comprises a HCVR comprising the amino acid sequence of SEQ ID NO: 16, and a LCVR comprising the amino acid sequence of SEQ ID NO: 28.

In some embodiments, the second antigen-binding domain of the bispecific antibody comprises: (a) three heavy chain complementarity determining regions (HCDR1, HCDR2 and HCDR3) contained within a heavy chain variable region (HCVR) comprising the amino acid sequence of SEQ ID NO: 20 of SEQ ID NO: 24; and (b) three light chain complementarity determining regions (LCDR1, LCDR2 and LCDR3) contained within a light chain variable region (LCVR) comprising the amino acid sequence of SEQ ID NO: 28. In some cases, the second antigen-binding domain comprises a HCDR1 comprising the amino acid sequence of SEQ ID NO: 21 or SEQ ID NO: 25, a HCDR2 comprising the amino acid sequence of SEQ ID NO: 22 or SEQ ID NO: 26, and a HCDR3 comprising the amino acid sequence of SEQ ID NO: 23 or SEQ ID NO: 27. In some cases, the second antigen-binding domain comprises a LCDR1 comprising the amino acid sequence of SEQ ID NO: 29, a LCDR2 comprising the amino acid sequence of SEQ ID NO: 30, and a LCDR3 comprising the amino acid sequence of SEQ ID NO: 31. In some embodiments, the second antigen-binding domain comprises a HCVR comprising the amino acid sequence of SEQ ID NO: 20 or SEQ ID NO: 24, and a LCVR comprising the amino acid sequence of SEQ ID NO: 28.

In any of the various embodiments discussed above or herein, the bispecific antibody may comprise a human IgG heavy chain constant region. In some cases, the human IgG heavy chain constant region is isotype IgG1. In some cases, the human IgG heavy chain constant region is isotype IgG4.

In any of the various embodiments discussed above or herein, the bispecific antibody may comprise a chimeric hinge that reduces Fcγ receptor binding relative to a wild-type hinge of the same isotype.

In any of the various embodiments discussed above or herein, the first heavy chain of the bispecific antibody or the second heavy chain of the bispecific antibody, but not both, may comprise a CH3 domain comprising a H435R (EU numbering) modification and a Y436F (EU numbering) modification.

In some embodiments, the bispecific antibody comprises a first heavy chain comprising the amino acid sequence of SEQ ID NO: 32.

In some embodiments, the bispecific antibody comprises a second heavy chain comprising the amino acid sequence of SEQ ID NO: 33.

In some embodiments, the bispecific antibody comprises a second heavy chain comprising the amino acid sequence of SEQ ID NO: 34.

In some embodiments, the bispecific antibody comprises a first heavy chain comprising the amino acid sequence of SEQ ID NO: 32, a second heavy chain comprising the amino acid sequence of SEQ ID NO: 33, and a common light chain comprising the amino acid sequence of SEQ ID NO: 35.

In some embodiments, the bispecific antibody comprises a first heavy chain comprising the amino acid sequence of SEQ ID NO: 32, a second heavy chain comprising the amino acid sequence of SEQ ID NO: 34, and a common light chain comprising the amino acid sequence of SEQ ID NO: 35.

In any of the various embodiments discussed above or herein, the antibody or antigen-binding fragment thereof that binds PD-1 comprises: (a) three heavy chain complementarity determining regions (HCDR1, HCDR2 and HCDR3) contained within a heavy chain variable region (HCVR) comprising the amino acid sequence of SEQ ID NO: 36; and (b) three light chain complementarity determining regions (LCDR1, LCDR2 and LCDR3) contained within a light chain variable region (LCVR) comprising the amino acid sequence of SEQ ID NO: 40. In some cases, the antibody or antigen-binding fragment thereof that binds PD-1 comprises a HCDR1 comprising the amino acid sequence of SEQ ID NO: 37, a HCDR2 comprising the amino acid sequence of SEQ ID NO: 38, and a HCDR3 comprising the amino acid sequence of SEQ ID NO: 39. In some cases, the antibody or antigen-binding fragment thereof that binds PD-1 comprises a LCDR1 comprising the amino acid sequence of SEQ ID NO: 41, a LCDR2 comprising the amino acid sequence of SEQ ID NO: 42, and a LCDR3 comprising the amino acid sequence of SEQ ID NO: 43. In some embodiments, the antibody or antigen-binding fragment thereof that binds PD-1 comprises a HCVR comprising the amino acid sequence of SEQ ID NO: 36, and a LCVR comprising the amino acid sequence of SEQ ID NO: 40. In some embodiments, the antibody or antigen-binding fragment thereof that binds PD-1 is an antibody comprising a heavy chain comprising the amino acid sequence of SEQ ID NO: 44 and a light chain comprising the amino acid sequence of SEQ ID NO: 45.

In any of the various embodiments discussed above or herein, the bispecific antibody or antigen-binding fragment thereof may be administered to the subject at a dose of from 0.03 mg to 1000 mg weekly. In some cases, the bispecific antibody or antigen-binding fragment thereof is administered to the subject at a dose of from 0.03 mg to 900 mg weekly. In some cases, the bispecific antibody or antigen-binding fragment thereof is administered to the subject at a dose of from 30 mg to 900 mg weekly. In some cases, the bispecific antibody or antigen-binding fragment thereof is administered to the subject at a dose of from 100 mg to 900 mg weekly. In some cases, the bispecific antibody or antigen-binding fragment thereof is administered to the subject at a dose of from 300 mg to 900 mg weekly.

In any of the various embodiments discussed above or herein, the bispecific antibody or antigen-binding fragment thereof may be administered to the subject at a dose of from 0.03 mg to 1000 mg once every three weeks. In some cases, the bispecific antibody or antigen-binding fragment thereof is administered to the subject at a dose of from 0.03 mg to 900 mg once every three weeks. In some cases, the bispecific antibody or antigen-binding fragment thereof is administered to the subject at a dose of from 30 mg to 900 mg once every three weeks. In some cases, the bispecific antibody or antigen-binding fragment thereof is administered to the subject at a dose of from 100 mg to 900 mg once every three weeks. In some cases, the bispecific antibody or antigen-binding fragment thereof is administered to the subject at a dose of from 300 mg to 900 mg once every three weeks.

In any of the various embodiments discussed above or herein, the antibody or antigen-binding fragment thereof that binds PD-1 may be administered to the subject at a dose of from 300 to 400 mg once every three weeks. In some cases, the antibody or antigen-binding fragment thereof that binds PD-1 is administered to the subject at a dose of 350 mg once every three weeks.

In any of the various embodiments discussed above or herein, the subject has stable disease, a partial response, or a complete response following administration of the bispecific antibody or antigen-binding fragment thereof for at least one week at a dose of from 0.03 mg to 900 mg in combination with the antibody or antigen-binding fragment thereof that binds PD-1.

In any of the various embodiments discussed above or herein, the subject may be further administered an IL-6R antagonist. In some cases, the IL-6R antagonist is an anti-IL-6R antibody. In some cases, the anti-IL-6R antibody is sarilumab or tocilizumab.

In any of the various embodiments discussed above or herein, the subject has:
at least a 50% decline in prostate specific antigen (PSA) levels in the subject;
at least a 55% decline in PSA levels in the subject;
at least a 60% decline in PSA levels in the subject;
at least a 65% decline in PSA levels in the subject;
at least a 70% decline in PSA levels in the subject;
at least a 75% decline in PSA levels in the subject;
at least a 80% decline in PSA levels in the subject;
at least a 85% decline in PSA levels in the subject;
at least a 90% decline in PSA levels in the subject;
at least a 95% decline in PSA levels in the subject;
at least a 96% decline in PSA levels in the subject;
at least a 97% decline in PSA levels in the subject;
at least a 98% decline in PSA levels in the subject;
at least a 99% decline in PSA levels in the subject;
a reduction in the size of at least one lesion that has a PSMA PET signal less than the PSMA PET signal in the subject's liver; and/or
a response in the subject following pseudo-progression,
following administration of the combination of a bispecific anti-PSMA x CD28 antibody (e.g., REGN5678) or antigen-binding fragment thereof and an anti-PD-1 antibody (e.g., cemiplimab) or antigen-binding fragment thereof.

The present disclosure also encompasses the use of the bispecific antibodies and/or the anti-PD-1 antibodies (and antigen-binding fragment of either) in the manufacture of a medicament for treating a PSMA-expressing cancer as set forth in any of the embodiments of the methods discussed above or herein The present disclosure also encompasses bispecific antibodies and/or anti-PD-1 antibodies (and antigen-binding fragments of either) for use in any of the embodiments of the methods discussed above or herein. The present disclosure also encompasses pharmaceutical compositions comprising the bispecific antibodies and/or anti-PD-1 antibodies (and antigen-binding fragments of either) for use in any of the embodiments of the methods discussed above or herein.

In one aspect, the present disclosure provides a method of treating a solid tumor in a subject in need thereof, comprising administering to the subject a combination of a bispecific antibody or antigen-binding fragment thereof comprising a first antigen-binding domain that specifically binds a tumor-associated antigen on the tumor cell, and a second antigen-binding domain that specifically binds human CD28 on a T cell, and an antibody or antigen-binding fragment thereof that specifically binds programmed death receptor-1 (PD-1).

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

Other embodiments of the present invention will become apparent from a review of the ensuing detailed description.

### BRIEF DESCRIPTION OF THE FIGURES

FIG. 1 illustrates an embodiment of a study flow diagram for QW dosing of REGN5678 in combination with Q3W dosing of cemiplimab, as discussed in Example 5. ¹Dose cohorts receiving QW dosing of REGN5678 may receive a 3-week monotherapy lead-in of REGN5678 QW followed by combination therapy of REGN5678 QW and cemiplimab Q3W. ²For some dose expansion cohorts, PSMA-PET/CT and optional whole body FDG-PET/CT will be performed at screening, 12-24 weeks post treatment initiation and upon disease progression. These PET/CT scans are optional for dose escalation patients. DLT=dose-limiting toxicity, PD=progressive disease, Q6W=every 6 weeks, Q12W=every 12 weeks.

FIG. 2 illustrates an embodiment of a study flow diagram for Q3W dosing of REGN5678 in combination with Q3W dosing of cemiplimab, as discussed in Example 5. ¹Dose cohorts with Q3W dosing interval may receive a 3-week monotherapy lead-in of REGN5678 followed by combination therapy of REGN5678 and cemiplimab. 2 For some dose expansion cohorts, PSMA-PET/CT and optional whole body FDG-PET/CT will be performed at screening, 12 to 24 weeks post-treatment initiation and upon disease progression. These PET/CT scans are optional for dose escalation patients. DLT=dose-limiting toxicity, PD=progressive disease, Q6W=every 6 weeks, Q12W=every 12 weeks.

FIGS. 3A, 3B, 3C and 3D are prostate-specific antigen (PSA) waterfall plots showing results (for first 33 patients) of combination administration of mAb1 (from 0.1 mg to 300 mg) and cemiplimab to patients with metastatic castration-resistant prostate cancer. Each figure shows the best percent change in PSA levels from baseline (baseline corresponds to PSA drawn immediately before first combo dose) for patients receiving varying doses of mAb1 in combination with cemiplimab. FIG. 3A shows all mAb1 dose levels together. FIG. 3B shows doses from 0.1 mg to 10 mg. FIG. 3C shows doses from 30 mg to 300 mg. FIG. 3D shows all doses in a comparison between doses of 30 mg vs. <30 mg. Dose levels (DL) correspond to those shown in Table 8. The DL number is shown above or below each bar in FIGS. 3A, 3B and 3C, and the bars corresponding to DL6-8 in FIG. 3D are identified with an asterisk.

FIGS. 4A and 4B are prostate-specific antigen (PSA) waterfall plots showing results (to date for first 35 patients) of combination administration of mAb1 (from 0.1 mg to 300 mg) and cemiplimab to patients with metastatic castration-resistant prostate cancer. Each figure shows the best percent change in PSA levels from baseline (baseline corresponds to PSA drawn immediately before first combo dose) for patients receiving varying doses of mAb1 in combination with cemiplimab. FIG. 4A shows doses from 0.1 mg to 10 mg. FIG. 4B shows doses from 30 mg to 300 mg. Dose levels (DL) correspond to those shown in Table 8. The DL number is shown above or below each bar in FIGS. 4A and 4B.

FIG. 5 illustrates declines in PSA levels in patients treated at dose level 8 (300 mg mAb1 and 350 mg cemiplimab). PSMA x CD28 corresponds to mAb1, and Libtayo™ is cemiplimab.

FIG. 6 illustrates an embodiment of a study flow diagram for QW dosing of REGN5678 in combination with Q3W dosing of cemiplimab and sarilumab, as discussed in Example 5. As shown, the dose of sarilumab will be 350 mg IV Q3W for a total of 12 weeks starting with the initial dose of REGN5678 in combination with cemiplimab (C1D1).

FIG. 7 illustrates an embodiment of a study flow diagram for Q3W dosing of REGN5678 in combination with Q3W dosing of cemiplimab and sarilumab, as discussed in Example 5. As shown, the dose of sarilumab will be 350 mg IV Q3W for a total of 12 weeks starting with the initial dose of REGN5678 in combination with cemiplimab (C1D1).

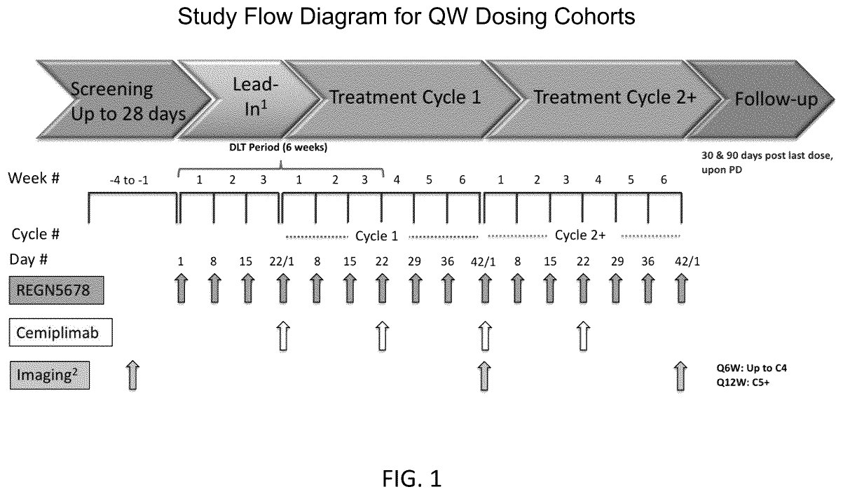

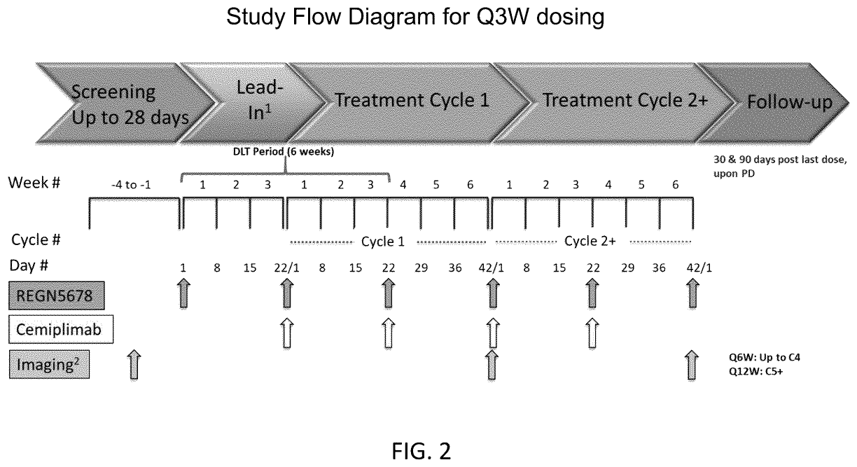

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims. Any embodiments or features of embodiments can be combined with one another, and such combinations are expressly encompassed within the scope of the present invention. Any specific value discussed above or herein may be combined with another related value discussed above or herein to recite a range with the values representing the upper and lower ends of the range, and such ranges are encompassed within the scope of the present disclosure.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Methods for Treating or Inhibiting the Growth of Cancers

The present disclosure includes methods for treating, ameliorating or reducing the severity of at least one symptom or indication, or inhibiting the growth of a cancer (e.g., metastatic castration-resistant prostate cancer) in a subject. The methods according to this aspect of the disclosure comprise administering a therapeutically effective amount of a bispecific antibody against PSMA and CD28 in combination with a therapeutically effective amount of an antibody or antigen-binding fragment thereof that specifically binds PD-1 to a subject in need thereof. As used herein, the terms “treat”, “treating”, or the like, mean to alleviate symptoms, eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, and/or to increase duration of survival of the subject.

As used herein, the expressions “a subject” or “a subject in need thereof” means a human or non-human mammal that exhibits one or more symptoms or indications of cancer, and/or who has been diagnosed with cancer, including a prostate cancer (e.g., metastatic castration-resistant prostate cancer) and who needs treatment for the same. In many embodiments, the term “subject” may be interchangeably used with the term “patient”. For example, a human subject may be diagnosed with a primary or a metastatic tumor and/or with one or more symptoms or indications including, but not limited to, enlarged lymph node(s), swollen abdomen, unexplained pain, unexplained weight loss, fever, night sweats, persistent fatigue, loss of appetite, and/or enlargement of spleen. The expression includes subjects with primary or established prostate tumors. In specific embodiments, the expression includes human subjects that have and need treatment for prostate cancer or another tumor expressing PSMA. In other specific embodiments, the expression includes subjects with PSMA+tumors (e.g., a tumor with PSMA expression as determined by flow cytometry). In certain embodiments, the expression “a subject in need thereof” includes patients with a prostate cancer that is resistant to or refractory to or is inadequately controlled by prior therapy (e.g., treatment with a conventional anti-cancer agent, including anti-androgen therapy). For example, the expression includes subjects who have been treated with chemotherapy, or anti-androgen therapy such as, for example, abiraterone, enzalutamide, apalutamide, or darolutamide. The expression also includes subjects with a prostate tumor for which conventional anti-cancer therapy is inadvisable, for example, due to toxic side effects. For example, the expression includes patients who have received one or more cycles of chemotherapy or other anti-cancer therapy with toxic side effects. In certain embodiments, the expression “a subject in need thereof” includes patients with a prostate tumor which has been treated but which has subsequently relapsed or metastasized. For example, patients with a prostate tumor that may have received treatment with one or more anti-cancer agents leading to tumor regression; however, subsequently have relapsed with cancer resistant to the one or more anti-cancer agents (e.g., castration-resistant prostate cancer) are treated with the methods of the present disclosure.

In certain embodiments, the methods of the present disclosure may be used to treat patients that have histologically or cytologically confirmed adenocarcinoma of the prostate without pure small cell carcinoma. In certain embodiments, the methods of the present disclosure may be used to treat patients that have metastatic castration-resistant prostate cancer with a prostate specific antigen (PSA) value of ≥4 ng/ml (e.g., 4 ng/ml, 4.5 ng/ml, 5 ng/ml, 5.5 ng/ml, 6 ng/ml, 6.5 ng/ml, 7 ng/ml, 7.5 ng/ml, 8 ng/ml, 8.5 ng/ml, 9 ng/ml, 9.5 ng/ml, or 10 ng/ml or more) prior to treatment with the bispecific antibody. In certain embodiments, the methods of the present disclosure may be used to treat patients with prostate cancer that has progressed within a period (e.g., 1 month, 2 months, 3 months, 4 months, 5 months, 6 months, 7 months, 8 months, 9 months, or more) prior to treatment with the bispecific antibody, wherein cancer progression is determined by, for example,: (a) a rising PSA level confirmed with an interval of 1 week (e.g., 1 week, 2 weeks, 3 weeks, 4 weeks, or more) between each assessment; (b) radiographic (e.g., PET/CT imaging) disease progression in soft tissue with or without a rise in PSA; and/or (c) radiographic (e.g., PET/CT imaging) disease progression in bone with an appearance of two or more bone lesions on bone scan with or without a rise in PSA. In certain embodiments, the methods of the present disclosure may be used to treat patients that have had an orchiectomy. In certain embodiments, the methods of the present disclosure may be used to treat patient that have or are receiving luteinizing hormone-releasing hormone (LHRH) agonist or antagonist therapy, and have a serum testosterone level of <50 ng/ml (e.g., from1 ng/ml to 49 ng/ml, about 45 ng/ml, about 40 ng/ml, about 35 ng/ml, about 30 ng/ml, about 25 ng/ml, about 20 ng/ml, about 15 ng/ml, about 10 ng/ml, or about 5 ng/ml) prior to treatment with the bispecific antibody.

In certain embodiments, the methods of the present disclosure are used in a subject with prostate cancer. The terms “tumor”, “cancer” and “malignancy” are interchangeably used herein. The term “prostate cancer”, as used herein, refers to tumors of the prostate, including metastatic tumors originating in the prostate.

According to certain embodiments, the present disclosure includes methods for treating, or delaying or inhibiting the growth of a tumor. In certain embodiments, the present disclosure includes methods to promote tumor regression. In certain embodiments, the present disclosure includes methods to reduce tumor cell load or to reduce tumor burden. In certain embodiments, the present disclosure includes methods to prevent tumor recurrence. The methods, according to this aspect of the disclosure, comprise administering a therapeutically effective amount of a bispecific anti-PSMA/anti-CD28 antibody or antigen-binding fragment thereof in combination with an anti-PD-1 antibody or antigen-binding fragment thereof to a subject in need thereof, wherein each antibody or fragment is administered to the subject in multiple doses, e.g., as part of a specific therapeutic dosing regimen. For example, the therapeutic dosing regimen may comprise administering one or more doses of an anti-PSMA x CD28 antibody or antigen-binding fragment thereof to the subject at a frequency of about once a day, once every two days, once every three days, once every four days, once every five days, once every six days, once a week, once every two weeks, once every three weeks, once every four weeks, once a month, once every two months, once every three months, once every four months, or less frequently. In certain embodiments, the anti-PSMA x anti-CD28 antibody or antigen-binding fragment thereof is administered once a week. In certain embodiments, the anti-PSMA x anti-CD28 antibody or antigen-binding fragment thereof is administered once every three weeks. In certain embodiments, the one or more doses of the anti-PD-1 antibody or antigen-binding fragment thereof are administered to the subject at a frequency of about once a day, once every two days, once every three days, once every four days, once every five days, once every six days, once a week, once every two weeks, once every three weeks, once every four weeks, once a month, once every two months, once every three months, once every four months, or less frequently. In certain embodiments, the anti-PD-1 antibody or antigen-binding fragment thereof is administered to the subject once every three weeks.

In certain embodiments, the present disclosure includes methods to inhibit, retard or stop tumor metastasis or tumor infiltration into peripheral organs. The methods, according to this aspect, comprise administering a therapeutically effective amount of a bispecific anti-PSMA/anti-CD28 antibody or antigen-binding fragment thereof in combination with an anti-PD-1 antibody or antigen-binding fragment thereof to a subject in need thereof.

In specific embodiments, the anti-PSMA/CD28 bispecific antibody or antigen-binding fragment thereof is administered to the subject prior to the anti-PD-1 antibody or antigen-binding fragment thereof. In some cases, the anti-PSMA/CD28 antibody or antigen-binding fragment thereof may be administered about 1 day, more than 1 day, more than 2 days, more than 3 days, more than 4 days, more than 5 days, more than 6 days, more than 7 days, 2 weeks, 3 weeks or more prior to the anti-PD-1 antibody or antigen-binding fragment thereof.

In certain embodiments, the methods of the present disclosure are used to treat a patient with a MRD-positive disease. Minimum residual disease (MRD) refers to small numbers of cancer cells that remain in the patient during or after treatment, wherein the patient may or may not show symptoms or signs of the disease. Such residual cancer cells, if not eliminated, frequently lead to relapse of the disease. The present disclosure includes methods to inhibit and/or eliminate residual cancer cells in a patient upon MRD testing. MRD may be assayed according to methods known in the art (e.g., MRD flow cytometry). The methods, according to this aspect of the disclosure, comprise administering a bispecific anti-PSMA/anti-CD28 antibody or antigen-binding fragment thereof in combination with an anti-PD-1 antibody or antigen-binding fragment thereof to a subject in need thereof.

The methods of the present disclosure, according to certain embodiments, comprise administering to a subject a therapeutically effective amount of a bispecific anti-PSMA/anti-CD28 antibody or antigen-binding fragment thereof in combination with an anti-PD-1 antibody or antigen-binding fragment thereof and, optionally, a third therapeutic agent. The third therapeutic agent may be an agent selected from the group consisting of, e.g., radiation, chemotherapy, surgery, a cancer vaccine, a PD-L1 inhibitor (e.g., an anti-PD-L1 antibody), a LAGS inhibitor (e.g., an anti-LAGS antibody), a CTLA-4 inhibitor (e.g., an anti-CTLA-4 antibody), a TIM3 inhibitor, a BTLA inhibitor, a TIGIT inhibitor, a CD47 inhibitor, an indoleamine-2,3-dioxygenase (IDO) inhibitor, a vascular endothelial growth factor (VEGF) antagonist, an Ang2 inhibitor, a transforming growth factor beta (TGF.beta.) inhibitor, an epidermal growth factor receptor (EGFR) inhibitor, an antibody to a tumor-specific antigen, a cytotoxin, a chemotherapeutic agent, anti-androgen therapy, an IL-6R inhibitor, an IL-4R inhibitor, an IL-10 inhibitor, a cytokine such as IL-2, IL-7, IL-21, and IL-15, an anti-inflammatory drug such as corticosteroids, and non-steroidal anti-inflammatory drugs, and a dietary supplement such as anti-oxidants. In certain embodiments, the antibodies may be administered in combination with therapy including a chemotherapeutic agent, radiation and surgery. As used herein, the phrase “in combination with” means that the antibodies are administered to the subject at the same time as, just before, or just after administration of the third therapeutic agent. In certain embodiments, the antibodies and the third therapeutic agent are administered in separate formulations.

In certain embodiments, the methods of the present disclosure comprise administering to a subject in need thereof a therapeutically effective amount of a bispecific anti-PSMA/anti-CD28 antibody or antigen-binding fragment thereof in combination with an anti-PD-1 antibody or antigen-binding fragment thereof. In certain embodiments, administration of the combination results in tumor growth inhibition by at least about 10%, about 20%, about 30%, about 40%, about 50%, about 60%, about 70% or about 80% as compared to an untreated subject. In certain embodiments, the administration of the combination leads to increased tumor regression, tumor shrinkage and/or disappearance. In certain embodiments, the administration of the combination leads to delay in tumor growth and development, e.g., tumor growth may be delayed by about 3 days, more than 3 days, about 7 days, more than 7 days, more than 15 days, more than 1 month, more than 3 months, more than 6 months, more than 1 year, more than 2 years, or more than 3 years as compared to an untreated subject. In certain embodiments, administration of the combination prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months relative to an untreated subject. In certain embodiments, administration of the combination increases progression-free survival or overall survival. In certain embodiments, administration of the combination increases response and duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% over an untreated subject. In certain embodiments, administration of the combination to a subject with prostate cancer leads to complete disappearance of all evidence of tumor cells (“complete response”). In certain embodiments, administration of the combination to a subject with prostate cancer leads to at least 30% or more decrease in tumor cells or tumor size (“partial response”). In certain embodiments, administration of the combination to a subject with prostate cancer leads to complete or partial disappearance of tumor cells/lesions including new measurable lesions. Tumor reduction can be measured by any of the methods known in the art, e.g., X-rays, positron emission tomography (PET), computed tomography (CT), magnetic resonance imaging (MRI), cytology, histology, or molecular genetic analyses. In some cases, PET/CT imaging can be performed using a radiotracer (e.g., ¹⁸F-DCFPyL) to detect lesions in patients with metastatic prostate cancer (e.g., mCRPC). In certain embodiments, administration of the bispecific antibody or antigen-binding fragment thereof and the anti-PD-1 antibody or antigen-binding fragment thereof produces a synergistic anti-tumor effect that exceeds the combined effects of the two agents when administered alone.

In certain cases, the response of a subject to therapy is categorized as a complete response (CR), a partial response (PR), progressive disease (PD), or as stable disease (SD). A CR is defined as disappearance of all target lesions, and a reduction in short axis of any pathological lymph nodes (whether target or non-target) to <10 mm (<1 cm). A PR is defined as an at least 30% decrease in the sum of the diameters of target lesions, taking as reference the baseline sum diameters. PD is defined as an at least 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm (0.5 cm). (Note: the appearance of one or more new lesions is also considered a progression). SD is defined as neither sufficient shrinkage to qualify for PR nor sufficient increase to qualify for PD, taking as reference the smallest sum diameters while on study.

### Anti-PD-1 Antibodies and Antigen-Binding Fragments Thereof

According to certain exemplary embodiments of the present disclosure, the methods comprise administering a therapeutically effective amount of an anti-PD-1 antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, the present disclosure includes methods comprising the use of bispecific antibodies wherein one arm of an immunoglobulin is specific for PD-1 or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab.sup.2 bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The antibodies used in the methods of the present disclosure may be human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the antibodies used in the methods of the present disclosure specifically bind PD-1. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PD-1, as used in the context of the present disclosure, includes antibodies that bind PD-1 or portion thereof with a K D of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PD-1 may, however, have cross-reactivity to other antigens, such as PD-1 molecules from other (non-human) species.

According to certain exemplary embodiments of the present disclosure, the anti-PD-1 antibody, or antigen-binding fragment thereof comprises a heavy chain variable region (HCVR), light chain variable region (LCVR), and/or complementarity determining regions (CDRs) comprising any of the amino acid sequences of the anti-PD-1 antibodies as set forth in U.S. Pat. No. 9,987,500. In certain exemplary embodiments, the anti-PD-1 antibody or antigen-binding fragment thereof that can be used in the context of the methods of the present disclosure comprises the heavy chain complementarity determining regions (HCDRs) of a heavy chain variable region (HCVR) comprising the amino acid sequence of SEQ ID NO: 36 and the light chain complementarity determining regions (LCDRs) of a light chain variable region (LCVR) comprising the amino acid sequence of SEQ ID NO: 40. According to certain embodiments, the anti-PD-1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 37; the HCDR2 comprises the amino acid sequence of SEQ ID NO: 38; the HCDR3 comprises the amino acid sequence of SEQ ID NO: 39; the LCDR1 comprises the amino acid sequence of SEQ ID NO: 41; the LCDR2 comprises the amino acid sequence of SEQ ID NO: 42; and the LCDR3 comprises the amino acid sequence of SEQ ID NO: 43. In yet other embodiments, the anti-PD-1 antibody or antigen-binding fragment thereof comprises an HCVR comprising SEQ ID NO: 36 and an LCVR comprising SEQ ID NO: 40. In certain embodiments, the methods of the present disclosure comprise the use of an anti-PD-1 antibody, wherein the antibody comprises a heavy chain comprising the amino acid sequence of SEQ ID NO: 44. In some embodiments, the anti-PD-1 antibody comprises a light chain comprising the amino acid sequence of SEQ ID NO: 45. An exemplary antibody comprising a HCVR comprising the amino acid sequence of SEQ ID NO: 36 and a LCVR comprising the amino acid sequence of SEQ ID NO: 40 is the fully human anti-PD-1 antibody known as REGN2810 (also known as cemiplimab, LIBTAYO®). According to certain exemplary embodiments, the methods of the present disclosure comprise the use of REGN2810, or a bioequivalent thereof. The term “bioequivalent”, as used herein, refers to anti-PD-1 antibodies or PD-1-binding proteins or fragments thereof that are pharmaceutical equivalents or pharmaceutical alternatives whose rate and/or extent of absorption do not show a significant difference with that of REGN2810 when administered at the same molar dose under similar experimental conditions, either single dose or multiple dose. In the context of the disclosure, the term refers to antigen-binding proteins that bind to PD-1 which do not have clinically meaningful differences with REGN2810 in their safety, purity and/or potency.

Other anti-PD-1 antibodies that can be used in the context of the methods of the present disclosure include, e.g., the antibodies referred to and known in the art as nivolumab (U.S. Pat. No. 8,008,449), pembrolizumab (U.S. Pat. No. 8,354,509), MEDI0608 (U.S. Pat. No. 8,609,089), pidilizumab (U.S. Pat. No. 8,686,119), or any of the anti-PD-1 antibodies as set forth in U.S. Pat. Nos. 6,808,710, 7,488,802, 8,168,757, 8,354,509, 8,779,105, or 8,900,587.

The anti-PD-1 antibodies used in the context of the methods of the present disclosure may have pH-dependent binding characteristics. For example, an anti-PD-1 antibody for use in the methods of the present disclosure may exhibit reduced binding to PD-1 at acidic pH as compared to neutral pH. Alternatively, an anti-PD-1 antibody of the disclosure may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to PD-1 at acidic pH as compared to neutral pH” is expressed in terms of a ratio of the K_{D} value of the antibody binding to PD-1 at acidic pH to the K_{D} value of the antibody binding to PD-1 at neutral pH (or vice versa). For example, an antibody or antigen-binding fragment thereof may be regarded as exhibiting “reduced binding to PD-1 at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### Bispecific Anti-PSMA x Anti-CD28 Antibodies

According to certain exemplary embodiments of the present disclosure, the methods comprise administering a therapeutically effective amount of a bispecific antibody that specifically binds CD28 and PSMA or antigen-binding fragment thereof. Such antibodies and fragments may be referred to herein as, e.g., “anti-PSMA/anti-CD28,” or “anti-PSMA x CD28” or “PSMA x CD28” bispecific antibodies or antigen-binding fragments thereof, or other similar terminology.

As used herein, the expression “bispecific antibody” refers to an immunoglobulin protein comprising at least a first antigen-binding domain and a second antigen-binding domain. In the context of the present disclosure, the first antigen-binding domain specifically binds a first antigen (e.g., PSMA), and the second antigen-binding domain specifically binds a second, distinct antigen (e.g., CD28). Each antigen-binding domain of a bispecific antibody comprises a heavy chain variable domain (HCVR) and a light chain variable domain (LCVR), each comprising three CDRs. In the context of a bispecific antibody, the CDRs of the first antigen-binding domain may be designated with the prefix “A” and the CDRs of the second antigen-binding domain may be designated with the prefix “B”. Thus, the CDRs of the first antigen-binding domain may be referred to herein as A-HCDR1, A-HCDR2, and A-HCDR3; and the CDRs of the second antigen-binding domain may be referred to herein as B-HCDR1, B-HCDR2, and B-HCDR3.

The first antigen-binding domain and the second antigen-binding domain are each connected to a separate multimerizing domain. As used herein, a “multimerizing domain” is any macromolecule, protein, polypeptide, peptide, or amino acid that has the ability to associate with a second multimerizing domain of the same or similar structure or constitution. In the context of the present disclosure, the multimerizing component is an Fc portion of an immunoglobulin (comprising a C_{H2}-C_{H3} domain), e.g., an Fc domain of an IgG selected from the isotypes IgG1, IgG2, IgG3, and IgG4, as well as any allotype within each isotype group.

Bispecific antibodies of the present disclosure typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

Any bispecific antibody format or technology may be used to make the bispecific antibodies of the present disclosure. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antibody. Specific exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab₂ bispecific formats (see, e.g., Klein et al. 2012, mAbs 4:6, 1-11, and references cited therein, for a review of the foregoing formats).

In the context of bispecific antibodies of the present disclosure, Fc domains may comprise one or more amino acid changes (e.g., insertions, deletions or substitutions) as compared to the wild-type, naturally occurring version of the Fc domain. For example, the disclosure includes bispecific antibodies comprising one or more modifications in the Fc domain that results in a modified Fc domain having a modified binding interaction (e.g., enhanced or diminished) between Fc and FcRn. In one embodiment, the bispecific antibody comprises a modification in a C_{H2} or a C_{H3} region, wherein the modification increases the affinity of the Fc domain to FcRn in an acidic environment (e.g., in an endosome where pH ranges from about 5.5 to about 6.0). Non-limiting examples of such Fc modifications are disclosed in US Patent Publication No. 20150266966, incorporated herein in its entirety.

The present disclosure also includes bispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second CH3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antibodies set forth herein comprises, from N- to C-terminus: [IgG4 C_{H}1]-[IgG4 upper hinge]-[IgG2 lower hinge]-[IgG4 CH2]-[IgG4 CH3]. Another example of a chimeric Fc domain that can be included in any of the antibodies set forth herein comprises, from N- to C-terminus: [IgG1 C_{H}1]-[IgG1 upper hinge]-[IgG2 lower hinge]-[IgG4 CH2]-[IgG1 CH3]. These and other examples of chimeric Fc domains or chimeric heavy chain constant regions that can be included in any of the antibodies of the present disclosure are described in US Patent Publication No. 20140243504, which is herein incorporated in its entirety. Chimeric Fc domains and chimeric heavy chain constant regions having these general structural arrangements, and variants thereof, can have altered Fc receptor binding, which in turn affects Fc effector function.

According to certain exemplary embodiments of the present disclosure, the bispecific anti-PSMA/anti-CD28 antibody, or antigen-binding fragment thereof comprises heavy chain variable regions (A-HCVR and B-HCVR), light chain variable regions (A-LCVR and B-LCVR), and/or complementarity determining regions (CDRs) comprising any of the amino acid sequences of the bispecific anti-PSMA/anti-CD28 antibodies as set forth in WO 2019/246514. In certain exemplary embodiments, the bispecific anti-PSMA/anti-CD28 antibody or antigen-binding fragment thereof that can be used in the context of the methods of the present disclosure comprises: (a) a first antigen-binding arm that specifically binds PSMA comprising the heavy chain complementarity determining regions (A-HCDR1, A-HCDR2 and A-HCDR3) of a heavy chain variable region (A-HCVR) comprising the amino acid sequence of SEQ ID NO: 1 and the light chain complementarity determining regions (A-LCDR1, A-LCDR2 and A-LCDR3) of a light chain variable region (A-LCVR) comprising the amino acid sequence of SEQ ID NO: 9; and (b) a second antigen-binding arm that specifically binds CD28 comprising the heavy chain CDRs (B-HCDR1, B-HCDR2 and B-HCDR3) of a HCVR (B-HCVR) comprising an amino acid sequence of SEQ ID NO: 5, and the light chain CDRs (B-LCDR1, B-LCDR2 and B-LCDR3) of a LCVR (B-LCVR) comprising the amino acid sequence of SEQ ID NO: 9. According to certain embodiments, the A-HCDR1 comprises the amino acid sequence of SEQ ID NO: 2; the A-HCDR2 comprises the amino acid sequence of SEQ ID NO: 3; the A-HCDR3 comprises the amino acid sequence of SEQ ID NO: 4; the A-LCDR1 comprises the amino acid sequence of SEQ ID NO: 10; the A-LCDR2 comprises the amino acid sequence of SEQ ID NO: 11; the A-LCDR3 comprises the amino acid sequence of SEQ ID NO: 12; the B-HCDR1 comprises the amino acid sequence of SEQ ID NO: 6; the B-HCDR2 comprises the amino acid sequence of SEQ ID NO: 7; and the B-HCDR3 comprises the amino acid sequence of SEQ ID NO: 8; and the B-LCDR1 comprises the amino acid sequence of SEQ ID NO: 10; the B-LCDR2 comprises the amino acid sequence of SEQ ID NO: 11; the B-LCDR3 comprises the amino acid sequence of SEQ ID NO: 12. In yet other embodiments, the bispecific anti-PSMA/anti-CD28 antibody or antigen-binding fragment thereof comprises: (a) a first antigen-binding arm comprising a HCVR (A-HCVR) comprising SEQ ID NO: 1 and a LCVR (A-LCVR) comprising SEQ ID NO: 9; and (b) a second antigen-binding arm comprising a HCVR (B-HCVR) comprising SEQ ID NO: 5, and a LCVR (B-LCVR) comprising SEQ ID NO: 9. In certain exemplary embodiments, the bispecific anti-PSMA x CD28 antibody comprises a PSMA-binding arm comprising a heavy chain comprising the amino acid sequence of SEQ ID NO: 13 and a light chain comprising the amino acid sequence of SEQ ID NO: 15, and a CD28-binding arm comprising a heavy chain comprising the amino acid sequence of SEQ ID NO: 14 and a light chain comprising the amino acid sequence of SEQ ID NO: 15.

In certain exemplary embodiments, the bispecific anti-PSMA/anti-CD28 antibody or antigen-binding fragment thereof that can be used in the context of the methods of the present disclosure comprises: (a) a first antigen-binding arm that specifically binds PSMA comprising the heavy chain complementarity determining regions (A-HCDR1, A-HCDR2 and A-HCDR3) of a heavy chain variable region (A-HCVR) comprising the amino acid sequence of SEQ ID NO: 16 and the light chain complementarity determining regions (A-LCDR1, A-LCDR2 and A-LCDR3) of a light chain variable region (A-LCVR) comprising the amino acid sequence of SEQ ID NO: 28; and (b) a second antigen-binding arm that specifically binds CD28 comprising the heavy chain CDRs (B-HCDR1, B-HCDR2 and B-HCDR3) of a HCVR (B-HCVR) comprising an amino acid sequence of SEQ ID NO: 20 or SEQ ID NO: 24, and the light chain CDRs (B-LCDR1, B-LCDR2 and B-LCDR3) of a LCVR (B-LCVR) comprising the amino acid sequence of SEQ ID NO: 28. According to certain embodiments, the A-HCDR1 comprises the amino acid sequence of SEQ ID NO: 17; the A-HCDR2 comprises the amino acid sequence of SEQ ID NO: 18; the A-HCDR3 comprises the amino acid sequence of SEQ ID NO: 19; the A-LCDR1 comprises the amino acid sequence of SEQ ID NO: 29; the A-LCDR2 comprises the amino acid sequence of SEQ ID NO: 30; the A-LCDR3 comprises the amino acid sequence of SEQ ID NO: 31; the B-HCDR1 comprises the amino acid sequence of SEQ ID NO: 21, or SEQ ID NO: 25; the B-HCDR2 comprises the amino acid sequence of SEQ ID NO: 22, or SEQ ID NO: 26; and the B-HCDR3 comprises the amino acid sequence of SEQ ID NO: 23, or SEQ ID NO: 27; and the B-LCDR1 comprises the amino acid sequence of SEQ ID NO: 29; the B-LCDR2 comprises the amino acid sequence of SEQ ID NO: 30; the B-LCDR3 comprises the amino acid sequence of SEQ ID NO: 31. In yet other embodiments, the bispecific anti-PSMA/anti-CD28 antibody or antigen-binding fragment thereof comprises: (a) a first antigen-binding arm comprising a HCVR (A-HCVR) comprising SEQ ID NO: 16 and a LCVR (A-LCVR) comprising SEQ ID NO: 28; and (b) a second antigen-binding arm comprising a HCVR (B-HCVR) comprising SEQ ID NO: 20 or SEQ ID NO: 24, and a LCVR (B-LCVR) comprising SEQ ID NO: 28. In certain exemplary embodiments, the bispecific anti-PSMA x CD28 antibody comprises a PSMA-binding arm comprising a heavy chain comprising the amino acid sequence of SEQ ID NO: 32 and a light chain comprising the amino acid sequence of SEQ ID NO: 35, and a CD28-binding arm comprising a heavy chain comprising the amino acid sequence of SEQ ID NO: 33 and a light chain comprising the amino acid sequence of SEQ ID NO: 35. In certain exemplary embodiments, the bispecific ant-PSMA x CD28 antibody comprises a PSMA-binding arm comprising a heavy chain comprising the amino acid sequence of SEQ ID NO: 32 and a light chain comprising the amino acid sequence of SEQ ID NO: 35, and a CD28-binding arm comprising a heavy chain comprising the amino acid sequence of SEQ ID NO: 34 and a light chain comprising the amino acid sequence of SEQ ID NO: 35.

### Combination Therapies

The methods of the present disclosure, according to certain embodiments, comprise administering to the subject an anti-PSMA/anti-CD28 bispecific antibody or antigen-binding fragment thereof in combination with an anti-PD-1 antibody or antigen-binding fragment thereof. In certain embodiments, the methods of the present disclosure comprise administering the antibodies for additive or synergistic activity to treat a PSMA-expressing cancer, preferably prostate cancer. In some embodiments, the combination of anti-PSMA x CD28 bispecific antibody (e.g., mAb1) and anti-PD-1 antibody (e.g., cemiplimab) produces a synergistic therapeutic effect in the treatment of metastatic castration-resistant prostate cancer. As used herein, the expression “in combination with” means that the anti-PSMA/anti-CD28 bispecific antibody or antigen-binding fragment thereof is administered before, after, or concurrent with the anti-PD-1 antibody or antigen-binding fragment thereof. The term “in combination with” also includes sequential or concomitant administration of anti-PD-1 antibody or antigen-binding fragment thereof and a bispecific anti-PSMA/anti-CD28 antibody or antigen-binding fragment thereof. For example, when administered “before” the bispecific anti-PSMA/anti-CD28 antibody or antigen-binding fragment thereof, the anti-PD-1 antibody or antigen-binding fragment thereof may be administered more than 72 hours, about 72 hours, about 60 hours, about 48 hours, about 36 hours, about 24 hours, about 12 hours, about 10 hours, about 8 hours, about 6 hours, about 4 hours, about 2 hours, about 1 hour, or about 30 minutes prior to the administration of the bispecific anti-PSMA/anti-CD28 antibody or antigen-binding fragment thereof. When administered “after” the bispecific anti-PSMA/anti-CD28 antibody or antigen-binding fragment thereof, the anti-PD-1 antibody or antigen-binding fragment thereof may be administered about 30 minutes, about 1 hour, about 2 hours, about 4 hours, about 6 hours, about 8 hours, about 10 hours, about 12 hours, about 24 hours, about 36 hours, about 48 hours, about 60 hours, about 72 hours, or more than 72 hours after the administration of the bispecific anti-PSMA/anti-CD28 antibody or antigen-binding fragment thereof. Administration “concurrent” with the bispecific anti-PSMA/anti-CD28 antibody or antigen-binding fragment thereof means that the anti-PD-1 antibody or antigen-binding fragment thereof is administered to the subject in a separate dosage form within less than 30 minutes (before, after, or at the same time) of administration of the bispecific anti-PSMA/anti-CD28 antibody or antigen-binding fragment thereof, or administered to the subject as a single combined dosage formulation comprising both the anti-PD-1 antibody or antigen-binding fragment thereof and the bispecific anti-PSMA/anti-CD28 antibody or antigen-binding fragment thereof.

In certain embodiments, the methods of the present disclosure comprise administration of a third therapeutic agent wherein the third therapeutic agent is an anti-cancer drug. In certain embodiments, the methods of the disclosure comprise administering an anti-PD-1 antibody or antigen-binding fragment thereof and an anti-PSMA/anti-CD28 bispecific antibody or antigen-binding fragment thereof in combination with radiation therapy, surgery or other anti-cancer therapy to generate long-term durable anti-tumor responses and/or enhance survival of patients with a PSMA-expressing cancer.

In some embodiments, the methods of the disclosure comprise administering radiation therapy prior to, concomitantly or after administering an anti-PD-1 antibody or antigen-binding fragment thereof and a bispecific anti-PSMA/anti-CD28 antibody or antigen-binding fragment thereof to a cancer patient. For example, radiation therapy may be administered in one or more doses to tumor lesions after administration of one or more doses of the antibodies. In some embodiments, radiation therapy may be administered locally to a tumor lesion to enhance the local immunogenicity of a patient's tumor (adjuvinating radiation) and/or to kill tumor cells (ablative radiation) after systemic administration of an anti-PD-1 antibody or antigen-binding fragment thereof and a bispecific anti-PSMA/anti-CD28 antibody or antigen-binding fragment thereof.

A clinical trial was conducted in which REGN5678 (mAb1) was administered in combination with the anti-PD-1 antibody cemiplimab in patients with advanced metastatic castration-resistant prostate cancer (CRPC) who have failed multiple anti-androgen therapies. Such patients generally have 1-2 years of life expectancy with limited treatment options. Metastatic CRPC is considered an immunologically “cold” tumor and is largely resistant to immune checkpoint therapy, with large trials of anti-PD-1 antibodies showing response rates in the single-digits. The anti-PSMAxCD28 costim bispecific of the present disclosure, REGN5678, was designed to enhance responsiveness in these types of tumor classes, such as prostate cancer, and essentially turn these cold tumors into hot tumors.

As detailed in the example set forth herein, patients were dosed weekly with REGN5678, and every three weeks with cemiplimab. The first dose of cemiplimab was not co-administered until week four, permitting a period of PSMAxCD28 lead-in to evaluate monotherapy safety and efficacy. The primary endpoints were safety, tolerability and pharmacokinetics. The secondary endpoint was objective response rate defined as a ≥50% decline of prostate-specific antigen (PSA) from baseline and/or tumor shrinkage. PSA is a protein produced by the prostate gland and is commonly used as a biomarker to diagnose and follow prostate cancer, as many mCRPC patients have disease limited to bone lesions and cannot be assessed by conventional RECIST criteria.

At the lowest 5 dose levels (cohorts 1-5), there was no evidence of any anti-tumor activity with 0 of 17 patients showing a PSA response and 16 of 17 patients showing an increase in PSA; there were no ≥Grade 3 (Gr3) immune-related adverse events (irAE) in these cohorts. The absence of anti-tumor activity among these patients was consistent with the approximate 6% response rate reported in other trials with anti-PD1 monotherapy.

At the next three dose levels (cohorts 6-8), evidence of dose-dependent anti-cancer activity was seen:
Cohort 6: 1 of 4 patients experienced a 100% decrease in PSA and a complete response (CR) based on RECIST criteria. The patient discontinued therapy due to a Gr3 irAE of the skin (that was considered to be a recurrence of a pre-existing condition, and has since resolved), but has maintained their 100% decrease in PSA and CR for 10 months of follow-up thus far.
Cohort 7: 3 of 8 patients showed a decrease in their PSA of 99%, 44% and 22%. Two of these three patients had Gr3 irAEs (aseptic encephalitis and seizure).
Cohort 8: 3 of 4 patients experienced large reductions in PSA within 6 weeks of starting combination treatment, including 2 patients with a 99% reduction and 1 with an 82% reduction. One patient with a 99% PSA reduction experienced a Gr3 case of acute inflammatory demyelinating polyradiculopathy.

In terms of safety, irAEs were correlated with anti-tumor activity with no ≥grade 3 irAEs among those who did not experience anti-tumor activity. Immune-related adverse events (also referred to herein as immune-mediated adverse events) can be managed with blockade of interleukin 6 receptor (IL-6R) or IL-6. For example, anti-IL-6R antibodies, such as sarilumab or tocilizumab, can be administered to patients in combination with the therapeutic agents discussed herein (e.g., anti-PSMAx CD28 bispecific antibodies and anti-PD-1 antibodies) to mitigate or prevent immune-mediated adverse events. No grade 4 irAEs, ≥grade 3 cytokine release syndrome, or treatment-related deaths have been observed in the trial as of data cutoff.

These data provide the first clinical evidence that a costimulatory bispecific antibody can synergistically combine with anti-PD-1, resulting in activity against a tumor class previously resistant to anti-PD-1 immunotherapy. More generally, the results observed in the present example indicate that costimulatory bispecific antibody therapies, wherein one arm binds a tumor antigen and the other arm provides a CD28 costimulatory immune cell signal, may provide a robust anti-tumor response, particularly when administered in combination with checkpoint inhibitors such as anti-PD-1 and anti-PD-L1 antibodies, in difficult-to-treat cancers.

In various embodiments, administration of the combination of anti-PSMA x CD28 bispecific antibody (e.g., mAb1) and anti-PD-1 antibody (e.g., cemiplimab) to a subject with mCRPC results in:
at least a 50% decline in prostate specific antigen (PSA) levels in the subject;
at least a 55% decline in PSA levels in the subject;
at least a 60% decline in PSA levels in the subject;
at least a 65% decline in PSA levels in the subject;
at least a 70% decline in PSA levels in the subject;
at least a 75% decline in PSA levels in the subject;
at least a 80% decline in PSA levels in the subject;
at least a 85% decline in PSA levels in the subject;
at least a 90% decline in PSA levels in the subject;
at least a 95% decline in PSA levels in the subject;
at least a 96% decline in PSA levels in the subject;
at least a 97% decline in PSA levels in the subject;
at least a 98% decline in PSA levels in the subject;
at least a 99% decline in PSA levels in the subject;
a reduction in the size of at least one lesion that has a PSMA PET signal less than the PSMA PET signal in the subject's liver; and/or
a response in the subject following pseudo-progression.

### Tumor Biopsy, Imaging, and CRS Monitoring/Management

The methods discussed in the present disclosure may further comprise tumor biopsies, imaging, and cytokine release syndrome (CRS) monitoring and management to evaluate efficacy and safety within individual subjects or populations of subjects.

### Tumor Biopsy

Patients with soft tissue disease may undergo a core or excisional biopsy from a soft tissue lesion if clinically accessible at screening and/or during treatment as discussed herein. For patients without soft tissue disease that is clinically accessible, a bone biopsy may be performed if feasible. Any available tissue from samples (e.g., formalin-fixed paraffin-embedded, or preserved in block for molecular extraction) collected at various time points, as well as archival specimens from previous treatment, in addition to clinical diagnostic uses, may be utilized for biomarker assays. Specifically, these samples may be assessed using in situ imaging with probes for gene targets relevant to REGN5678 (PSMA, CD28) and cemiplimab (PD-L1), as well as markers of immune activation, suppression, and function, and tumor cell phenotype. As discussed in Example 5, expression of the therapeutic pathway targets of REGN5678 and cemiplimab is an exploratory endpoint.

Tumor tissue biopsies, if available, may also be subjected to gene expression profiling (using RNA sequencing or other methods) as a measure of composite tumor microenvironmental phenotype, whole exome sequencing or other mutational profiling, and targeted study of gene variants (tumor mutations) such as those affecting DNA repair pathways. They may also be profiled using next-generation sequencing for the T cell receptor repertoire, as a measure of tumor-associated T cell clonal proliferation.

### Imaging

Prostate-specific membrane antigen (PSMA) PET/CT has been shown to provide a sensitive measure of both PSMA expression and tumor burden in prostate cancer patients. It allows detection of more tumor lesions and greater specificity than conventional imaging modalities used in combination for prostate cancer, such as CT, MRI and bone scan, thereby greatly improves the effectiveness of tumor response assessment and treatment strategy decision-making.

Fluorine F 18 DCFPyL (¹⁸F-DCFPyL) is a radiolabeled small molecule that binds to the extracellular domain of PSMA with high affinity. Data from enzyme inhibition assays has shown that DCFPyL binds competitively to PSMA expressing LNCaP cells with a Ki of 1.1 nM. ¹⁸F-DCFPyL has been tested in multiple phase 1 to 3 studies and found to be well tolerated in prostate cancer patients. Biodistribution following administration of ¹⁸F-DCFPyL injection and optimal imaging time point were determined and radiation dose used was within limit for diagnostic radiotracers for PET. Physiologic accumulation of ¹⁸F-DCFPyL was found to correspond to the distribution of PSMA expressing organs. Accumulation in primary tumor and metastatic lesions was very high, suggesting that ¹⁸F-DCFPyL injection can be used to detect residual tumor as well as regional or distant metastases with high sensitivity and specificity. According, the methods discussed herein may include using ¹⁸F-DCFPyL PSMA PET/CT for assessing whole body tumor burden in mCRPC patients and the anti-tumor activity of the REGN5678 and cemiplimab combination.

### CRS Monitoring and Management

Cytokine release has been observed with superagonist anti-CD28 bivalent antibodies, bsAbs, and similar molecules. Cytokine release syndrome (CRS) has often resulted in clinical symptoms during infusion or within hours to days of infusion. In a clinical study of 6 patients treated with a bivalent anti-CD28 superagonist antibody (TGN1412), life-threatening CRS occurred acutely, and patients became critically ill within 12 to 16 hours. Prior experience with bispecific antibodies targeting a tumor antigen and CD3 has shown that when CRS occurred, the events were most prominent following the first 1 or 2 weekly doses of study treatment and were typically transient, even when higher doses were administered in subsequent weeks. This has also been observed in combination with cemiplimab. CRS typically occurs more frequently with the first 2 weekly doses for any given patient and decreases in frequency upon subsequent exposure. Based on these findings, the risk of an initial episode of CRS occurring after third dose or later is considered to be low.

Subcutaneous administration of bispecific antibodies has recently been evaluated in preclinical and early-phase clinical studies. Subcutaneous administration of a bispecific antibody targeting a tumor antigen and CD3 was tolerated without severe CRS events (no Grade CRS) in a B-cell tumor. In cynomolgus monkeys, SC administration of the same bispecific antibody resulted in lower Cₘₐₓ, delayed Tₘₐₓ, and lower plasma cytokine levels compared to IV administration. The methods discussed herein may include measures to address potential safety issues resulting from cytokine release, including:
(1) Cytokine monitoring;
(2) Use of anti-IL-6 pathway therapies (e.g., sarilumab or tocilizumab) and corticosteroids for management of CRS; and
(3) Provisions for premedication and use of lower dose at initial dosing visits before stepping up to the full dose at the dose level (DL) in the event of observed CRS.

Patients who develop symptoms consistent with severe CRS, including but not limited to, persistent fevers, neurologic disorders (including mental status changes, obtundation, and seizures), clinical signs of toxicity (hypotension requiring at least 1 IV vasoactive pressor or hypoxia [PO₂<90%]) may be considered for pharmacologic intervention with anti-IL-6 pathway therapies (e.g., sarilumab or tocilizumab) and/or high dose steroids. Such additions to the methods discussed herein are contemplated by this disclosure.

Corticosteroids may also be utilized in the management of CRS, particularly in cases with neurologic symptoms. In general, corticosteroids should be used when: 1) IRR/CRS does not respond adequately to anti-IL-6 pathway therapies (e.g., sarilumab or tocilizumab), or 2) anti-IL-6 pathway therapies are not in the best interest of the patient.

### Pharmaceutical Compositions and Administration

The present disclosure includes methods which comprise administering a bispecific anti-PSMA/anti-CD28 antibody or antigen-binding fragment thereof in combination with an anti-PD-1 antibody or antigen-binding fragment thereof to a subject wherein the antibody or antibodies (or fragments) are contained within separate or a combined (single) pharmaceutical composition. The pharmaceutical compositions of the disclosure may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262: 4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, or by injection, and may be administered together with other biologically active agents.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL), to name only a few.

In certain situations, the pharmaceutical composition can be delivered in a controlled release system. In one embodiment, a pump may be used. In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent. The injection thus prepared is preferably filled in an appropriate ampoule.

Advantageously, the pharmaceutical compositions for use described above are prepared into dosage forms in a unit dose suited to fit a dose of the active ingredients. Such dosage forms in a unit dose include, for example, a vial or a prefilled syringe.

### Administration Regimens

The present disclosure includes methods comprising administering to a subject a bispecific anti-PSMA x CD28 antibody or antigen-binding fragment thereof in combination with an anti-PD-1 antibody or antigen-binding fragment thereof at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

According to certain embodiments of the present disclosure, multiple doses of a bispecific anti-PSMA/anti-CD28 antibody or antigen-binding fragment thereof in combination with an anti-PD-1 antibody or antigen-binding fragment thereof may be administered to a subject over a defined time course. The methods according to this aspect of the disclosure comprise sequentially administering to a subject one or more doses of a bispecific anti-PSMA/anti-CD28 antibody or antigen-binding fragment thereof in combination with one or more doses of an anti-PD-1 antibody or antigen-binding fragment thereof. As used herein, “sequentially administering” means that each dose of the antibody is administered to the subject at a different point in time, e.g., on different days separated by a predetermined interval (e.g., hours, days, weeks or months). The present disclosure includes methods which comprise sequentially administering to the patient a single initial dose of an antibody (or fragment), followed by one or more secondary doses of the antibody (or fragment), and optionally followed by one or more tertiary doses of the antibody (or fragment).

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antibody or antigen-binding fragment thereof (anti-PD-1 antibody or bispecific antibody). In certain embodiments, however, the amount contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered 1/2 to 14 (e.g., 1/2, 1, 11/2, 2, 21/2, 3, 31/2, 4, 41/2, 5, 51/2, 6, 61/2, 7, 71/2, 8, 81/2, 9, 91/2, 10, 101/2, 11, 111/2, 12, 121/2, 13, 131/2, 14, 141/2, or more) weeks after the immediately preceding dose. The phrase “the immediately preceding dose,” as used herein, means, in a sequence of multiple administrations, the dose of a bispecific anti-PSMA/anti-CD28 or antigen-binding fragment thereof (and/or anti-PD-1 antibody or antigen-binding fragment thereof) which is administered to a patient prior to the administration of the very next dose in the sequence with no intervening doses.

The methods according to this aspect of the disclosure may comprise administering to a patient any number of secondary and/or tertiary doses of bispecific anti-PSMA/anti-CD28 antibody or antigen-binding fragment thereof and an anti-PD-1 antibody or antigen-binding fragment thereof. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1, 2 or 3 weeks (e.g., 1 week or 3 weeks) after the immediately preceding dose. Similarly, in embodiments involving multiple tertiary doses, each tertiary dose may be administered at the same frequency as the other tertiary doses. For example, each tertiary dose may be administered to the patient 1 to 4 weeks (e.g., 1 week or 3 weeks) after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In certain embodiments, one or more doses of a bispecific anti-PSMA/anti-CD28 antibody or antigen-binding fragment thereof, and an anti-PD-1 antibody or antigen-binding fragment thereof are administered at the beginning of a treatment regimen as “induction doses” on a more frequent basis (twice a week, once a week, once in 2 weeks, or once in 3 weeks) followed by subsequent doses (“consolidation doses” or “maintenance doses”) that are administered on the same or a less frequent basis (e.g., once in 4-12 weeks).

The present disclosure includes methods comprising sequential administration of a bispecific anti-PSMA/anti-CD28 antibody or antigen-binding fragment thereof in combination with an anti-PD-1 antibody or antigen-binding fragment thereof to a patient to treat prostate cancer (e.g., metastatic castration-resistant prostate cancer). In some embodiments, the present methods comprise administering one or more doses of a bispecific anti-PSMA/anti-CD28 antibody or antigen-binding fragment thereof, preceded by or followed by one or more doses of an anti-PD-1 antibody or antigen-binding fragment thereof. In certain embodiments, the present methods comprise administering several doses (e.g., once weekly over a 3 week lead-in period) of a bispecific anti-PSMA/anti-CD28 antibody, followed by administration of one or more doses of an anti-PD-1 antibody or antigen-binding fragment thereof and one or more additional doses of the anti-PSMA x CD28 bispecific antibody or antigen-binding fragment thereof. In some embodiments, one or more doses of about 100 to 600 mg of an anti-PD-1 antibody or antigen-binding fragment thereof may be administered along with one or more doses of about 0.1 mg/kg to about 20 mg/kg (e.g., 0.01 to 1000 mg) of the bispecific antibody or antigen-binding fragment thereof to inhibit tumor growth and/or to prevent tumor recurrence in a subject with prostate cancer. In some embodiments, the bispecific antibody or antigen-binding fragment thereof in combination with the anti-PD-1 antibody or antigen-binding fragment thereof results in increased anti-tumor efficacy (e.g., greater inhibition of tumor growth, or increased prevention of tumor recurrence as compared to an untreated subject. In some embodiments, the bispecific antibody or antigen-binding fragment thereof is administered before, after or concurrently with the anti-PD-1 antibody or antigen-binding fragment thereof. In certain embodiments, the bispecific antibody or antigen-binding fragment thereof and the anti-PD-1 antibody or antigen-binding fragment thereof are administered in separate dosage formulations.

### Dosage

The amount of bispecific anti-PSMA/anti-CD28 antibody or antigen-binding fragment thereof, and anti-PD-1 antibody or antigen-binding fragment thereof, administered to a subject according to the methods of the present disclosure is, generally, a therapeutically effective amount.

As used herein, the phrase “therapeutically effective amount” means an amount of antibody (anti-PD-1 antibody or bispecific anti-PSMA/anti-CD28 antibody) or antigen-binding fragment thereof that results in one or more of: (a) a reduction in the severity or duration of a symptom of a cancer (e.g., prostate cancer); (b) inhibition of tumor growth, or an increase in tumor necrosis, tumor shrinkage and/or tumor disappearance; (c) delay in tumor growth and development; (d) inhibit or retard or stop tumor metastasis; (e) prevention of recurrence of tumor growth; (f) increase in survival of a subject with cancer (e.g., prostate cancer); and/or (g) a reduction in the use or need for conventional anti-cancer therapy (e.g., reduced or eliminated use of chemotherapeutic or cytotoxic agents) as compared to an untreated subject.

In the case of a bispecific anti-PSMA/anti-CD28 antibody or antigen-binding fragment thereof, a therapeutically effective amount can be from about 0.01 milligrams (mg) to about 2000 mg, e.g., about 0.1 mg, about 0.2 mg, about 0.3 mg, about 0.5 mg, about 1 mg, about 3 mg, about 5 mg, about 10 mg, about 20 mg, about 30 mg, about 40 mg, about 50 mg, about 75 mg, about 100 mg, about 125 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of the bispecific anti-PSMA/anti-CD28 antibody or antigen-binding fragment thereof. In certain embodiments, 0.03 mg, 0.1 mg, 0.3 mg, 1 mg, 3 mg, 10 mg, 30 mg, 100 mg, 300 mg, or 900 mg of the bispecific anti-PSMA x anti-CD28 antibody or antigen-binding fragment thereof is administered (e.g., once weekly or once every three weeks) to the subject to treat a PSMA-expressing cancer or prostate cancer (e.g., metastatic and/or castration-resistant prostate cancer).

In the case of an anti-PD-1 antibody or antigen-binding fragment thereof, a therapeutically effective amount can be from about 100 mg to about 600 mg, e.g., about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-PD-1 antibody or antigen-binding fragment thereof. In certain embodiments, 300 mg to 400 mg of the anti-PD-1 antibody or antigen-binding fragment thereof is administered (e.g., once every three weeks) to the subject in combination with the bispecific antibody or antigen-binding fragment thereof to treat a PSMA-expressing cancer or prostate cancer (e.g., metastatic and/or castration-resistant prostate cancer). In certain embodiments, 350 mg of an anti-PD-1 antibody or antigen-binding fragment thereof is administered (e.g., once every three weeks) to the subject in combination with the bispecific antibody or antigen-binding fragment thereof to treat a PSMA-expressing cancer or prostate cancer (e.g., metastatic and/or castration-resistant prostate cancer).

The amount of bispecific anti-PSMA/anti-CD28 antibody or antigen-binding fragment thereof and anti-PD-1 antibody or antigen-binding fragment thereof contained within the individual doses may be expressed in terms of milligrams of antibody per kilogram of subject body weight (i.e., mg/kg). In certain embodiments, the bispecific anti-PSMA/anti-CD28 antibody or antigen-binding fragment thereof may be administered at a dose of about 0.1 mg/kg to about 20 mg/kg of a patient's body weight, and the anti-PD-1 antibody or antigen-binding fragment thereof may be administered at dose of about 2 mg/kg to about 20 mg/kg of a patient's body weight.

A summary of the sequences and the corresponding SEQ ID NOs referenced herein is shown in Table 1, below.

TABLE 1
Summary of Sequences

| SEQ ID NO: | Description |
|---|---|
| 1 | Anti-PSMA Heavy Chain Variable Region (-001) |
| 2 | Anti-PSMA HCDR1 (-001) |
| 3 | Anti-PSMA HCDR2 (-001) |
| 4 | Anti-PSMA HCDR3 (-001) |
| 5 | Anti-CD28 Heavy Chain Variable Region (-001) |
| 6 | Anti-CD28 HCDR1 (-001) |
| 7 | Anti-CD28 HCDR2 (-001) |
| 8 | Anti-CD28 HCDR3 (-001) |
| 9 | Anti-PSMA and Anti-CD28 |
| | Light Chain Variable Region (-001) |
| 10 | Anti-PSMA and Anti-CD28 LCDR1 (-001) |
| 11 | Anti-PSMA and Anti-CD28 LCDR2 (-001) |
| 12 | Anti-PSMA and Anti-CD28 LCDR3 (-001) |
| 13 | Anti-PSMA Heavy Chain (-001) |
| 14 | Anti-CD28 Heavy Chain (-001) |
| 15 | Anti-PSMA and Anti-CD28 Light Chain (-001) |
| 16 | Anti-PSMA Heavy Chain Variable Region |
| | (-002 and -003) |
| 17 | Anti-PSMA HCDR1 (-002 and -003) |
| 18 | Anti-PSMA HCDR2 (-002 and -003) |
| 19 | Anti-PSMA HCDR3 (-002 and -003) |
| 20 | Anti-CD28 Heavy Chain Variable Region (-002) |
| 21 | Anti-CD28 HCDR1 (-002) |
| 22 | Anti-CD28 HCDR2 (-002) |
| 23 | Anti-CD28 HCDR3 (-002) |
| 24 | Anti-CD28 Heavy Chain Variable Region (-003) |
| 25 | Anti-CD28 HCDR1 (-003) |
| 26 | Anti-CD28 HCDR2 (-003) |
| 27 | Anti-CD28 HCDR3 (-003) |
| 28 | Anti-PSMA and Anti-CD28 Light Chain |
| | Variable Region (-002 and -003) |
| 29 | Anti-PSMA and Anti-CD28 LCDR1 (-002 and -003) |
| 30 | Anti-PSMA and Anti-CD28 LCDR2 (-002 and -003) |
| 31 | Anti-PSMA and Anti-CD28 LCDR3 (-002 and -003) |
| 32 | Anti-PSMA Heavy Chain (-002 and -003) |
| 33 | Anti-CD28 Heavy Chain (-002) |
| 34 | Anti-CD28 Heavy Chain (-003) |
| 35 | Anti-MUC16 and Anti-CD28 Light Chain |
| | (-002 and -003) |
| 36 | Anti-PD-1 Heavy Chain Variable Region |
| 37 | Anti-PD-1 HCDR1 |
| 38 | Anti-PD-1 HCDR2 |
| 39 | Anti-PD-1 HCDR3 |
| 40 | Anti-PD-1 Light Chain Variable Region |
| 41 | Anti-PD-1 LCDR1 |
| 42 | Anti-PD-1 LCDR2 |
| 43 | Anti-PD-1 LCDR3 |
| 44 | Anti-PD-1 Heavy Chain |
| 45 | Anti-PD-1 Light Chain |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Bispecific Antibodies that Bind Prostate-Specific Membrane Antigen (PSMA) and CD28

Bispecific antibodies comprising an anti-PSMA-specific binding domain and an anti-CD28-specific binding domain were constructed using standard methodologies, wherein the anti-PSMA antigen binding domain and the anti-CD28 antigen binding domain each comprise different, distinct HCVRs paired with a common LCVR. In some instances the bispecific antibodies were constructed utilizing a heavy chain from an anti-CD28 antibody, a heavy chain from an anti-PSMA antibody and a common light chain (See Table 2).

The bispecific antibodies created in accordance with the present Example comprise two separate antigen-binding domains (i.e., binding arms). The first antigen-binding domain comprises a heavy chain variable region derived from an anti-CD28 antibody (“CD28-VH”), and the second antigen-binding domain comprises a heavy chain variable region derived from an anti-PSMA antibody (“PSMA-VH”). Both the anti-PSMA and the anti-CD28 share a common light chain. The CD28-VH/PSMA-VH pairing creates antigen-binding domains that specifically recognize CD28 on T cells and PSMA on tumor cells.

A summary of the component parts of the antigen-binding domains of the various anti-PSMAxCD28 bispecific antibodies constructed is set forth in Table 3. The corresponding CDR sequences and full-length heavy and light chain sequences are identified in Table 1 (with reference to the “-001,” “-002,” and “-003” bispecific antibodies of Table 3).

TABLE 2
Summary of Component Parts of Anti-PSMAxCD28 Bispecific Antibodies

| | Anti-PSMA | Anti-CD28 | |
|---|---|---|---|
| | Antigen-Binding | Antigen-Binding | |
| | Domain | Domain | Common |
| | Heavy Chain | Heavy Chain | Light Chain |
| Bispecific Antibody Identifier | Variable Region | Variable Region | Variable Region |
| BSPSMA/CD28-001 | (SEQ ID NO: 1) | (SEQ ID NO: 5) | (SEQ ID NO: 9) |
| (also referred to as mAb1 or | | | |
| REGN5678) | | | |
| BSPSMA/CD28-002 | (SEQ ID NO: 16) | (SEQ ID NO: 20) | (SEQ ID NO: 28) |
| BSPSMA/CD28-003 | (SEQ ID NO: 16) | (SEQ ID NO: 24) | (SEQ ID NO: 28) |

### Example 2: Surface Plasmon Resonance Derived Binding Affinities and Kinetic Constants of anti-PSMAxCD28 Bispecific Antibodies

In order to determine the binding kinetics of anti-PSMAxCD28 bispecific antibodies, surface plasmon resonance derived binding affinities and kinetic constants of anti-PSMAxCD28 bispecific were determined.

Binding Kinetics of anti-PSMAxCD28 Bispecific Antibodies to PSMA: Equilibrium dissociation constants (K_{D} values) for 6h.hPSMA (recombinant Human PSMA/FOLH1 Protein, R&D, Catalog #4234-ZN) binding to purified anti-PSMAxCD28 bispecific antibodies were determined using a real-time surface plasmon resonance biosensor using a Biacore T-200 instrument. The CM5 Biacore sensor surface was derivatized by amine coupling with a monoclonal mouse anti-human Fc antibody to capture purified anti-PSMAxCD28 bispecific antibodies.

This Biacore binding study was performed in a buffer composed of 0.01M HEPES pH 7.4, 0.15M NaCl, 0.5mM MgCl₂, 1.0 mM CaCl₂, 0.05% v/v Surfactant P20 (HBS-P++ running buffer). Different concentrations of hPSMA with an N-terminal polyhistidine tag (6h.hPSMA, R&D) were prepared in HBS-P++ running buffer, ranging from 10 nM to 0.4 nM with serially 3-fold dilutions for anti-PSMAxCD28 bispecific antibodies.

The different concentrations of 6h.hPSMA were injected over the monoclonal antibody captured surface at a flow rate of 50 μL/minute. Association of 6h.hPSMA to the captured monoclonal antibody was monitored for 3 minutes and the dissociation of 6h.hPSMA in HBS-P++ running buffer was monitored for 10 minutes. Kinetic association (kₐ) and dissociation (k_{d}) rate constants were determined by fitting the real-time sensorgrams to a 1:1 binding model using Scrubber 2.0c curve fitting software (BioLogic Software). Binding dissociation equilibrium constants (K_{D}) and dissociative half-lives (t1/2) were calculated from the kinetic rate constants as: K_{D}(M)=k_{d}/kₐ, and t1/2 (min)=0.693/k_{d}/60

Binding kinetic parameters for 6h.hPSMA binding to purified monoclonal antibodies at are shown below in Table 3.

TABLE 3
Biacore Binding Affinities of Monoclonal Antibodies to PSMA at 25° C.

| Antibody ID | ka (1/Ms) | kd (1/s) | K_{D} (M) | T½ (min) |
|---|---|---|---|---|
| BSPSMA/CD28-001 | 1.96E+05 | 4.92E−05 | 2.51E−10 | 234.6 |
| BSPSMA/CD28-002 | TBD | TBD | TBD | TBD |
| BSPSMA/CD28-003 | 2.80E+05 | 3.85E−05 | 1.37E−10 | 300.4 |

TBD: not tested

Binding kinetic parameters for 6h.hPSMA binding to one purified exemplary monoclonal bispecific antibody at 37° C. are shown below in Table 4. One (1) RU (response unit) represents 1 pg of protein per mm², as defined by the manufacturer.

TABLE 4
Biacore Binding Affinities of Monoclonal Antibody to PSMA at 37° C.

| | mAb | 10 nM | | | | |
|---|---|---|---|---|---|---|
| | Capture | hPSMA.6H | | | | |
| Antibody ID | (RU) | Bind (RU) | Ka (1/Ms) | Kd (1/s) | K_{D} (M) | t½ |
| BSPSMA/CD28-001 | 256.8 ± 0.9 | 43.5 | 2.00E+05 | 7.93E−05 | 3.96E−10 | 145.7 |
| | mAb | 20 nM | | | | |
| | Capture | hPSMA.6H | | | | |
| Antibody ID | (RU) | Bind (RU) | Ka (1/Ms) | Kd (1/s) | K_{D} (M) | t½ |
| BSPSMA/CD28-001 | 189.7 ± 1.6 | 73.7 | 2.93E+05 | 6.36E+05 | 2.17E−10 | 181.6 |

Binding Kinetics of anti-PSMAxCD28 Bispecific Antibodies to CD28: Equilibrium dissociation constants (K_{D} values) for hCD28.mmh binding to purified monoclonal antibodies were determined using a real-time surface plasmon resonance biosensor using a Biacore T-200 instrument. The CM5 Biacore sensor surface was derivatized by amine coupling with a monoclonal mouse anti-human Fc antibody to capture purified anti-PSMAxCD28 bispecific antibodies.

Different concentrations of hCD28.mmh were injected over the monoclonal antibody captured surface at a flow rate of 50 μL/minute. Association of hCD28.mmh to the captured monoclonal antibody was monitored for 5 minutes and the dissociation of hCD28.mmh in HBS-P++ running buffer was monitored for 10 minutes. Kinetic association (kₐ) and dissociation (k_{d}) rate constants were determined by fitting the real-time sensorgrams to a 1:1 binding model using Scrubber 2.0c curve fitting software. Binding dissociation equilibrium constants (K_{D}) and dissociative half-lives (t1/2) were calculated from the kinetic rate constants as: K_{D}(M)=k_{d}/kₐ, and t1/2 (min)=0.693/k_{d}/60

Binding kinetic parameters for hCD28.mmh binding to purified anti-PSMAxCD28 bispecific antibodies at 25° C. are shown below in Table 5.

TABLE 5
Biacore Binding Affinities of Monoclonal Antibodies to CD28 at 25° C.

| Antibody ID | ka (1/Ms) | kd (1/s) | K_{D} (M) | T½ (min) |
|---|---|---|---|---|
| BSPSMA/CD28-001 | 2.26E+04 | 3.26E−03 | 1.44E−07 | 3.5 |
| BSPSMA/CD28-002 | TBD | TBD | TBD | TBD |
| BSPSMA/CD28-003 | 6.79E+03 | 1.41E−03 | 2.07E−07 | 8.2 |

TBD: not tested

Binding kinetic parameters for hCD28.mmh binding to purified anti-PSMAxCD28 bispecific antibody 37° C. are shown below in Table 6.

TABLE 6
Biacore Binding Affinities of Monoclonal Antibodies to CD28 at 37° C.

| | mAb | 400 nM | | | | |
|---|---|---|---|---|---|---|
| | Capture | hCD28.6H | | | | |
| Antibody ID | (RU) | Bind (RU) | Ka (1/Ms) | Kd (1/s) | K_{D} (M) | t½ |
| BSPSMA/CD28-001 | 1576.3 ± 5.0 | 98.9 | 2.46E+04 | 6.70E−03 | 2.72E−07 | 1.7 |

### Example 3. Cell Binding of Anti-PSMAxCD28 Bispecific Antibodies to PSMA and CD28

In order to evaluate the ability of these antibodies (anti-PSMAxCD28 antibodies) to bind specifically to the cell-surface proteins, in vitro binding assays were performed.

The Binding of PSMAxCD28 bispecific antibodies to the surface of Human T cells was tested by flow cytometry. BSPSMA/CD28-001 bound to all T cells with an EC50 value of 4.80×10⁻⁸ M, and bound to both CD4+ and CD8+ T cells, with EC50 values of 5.09×10⁻⁸ M and 5.89×10⁻⁸ M, respectively. BSPSMA/CD28-003 bound weakly to all T cells with an EC50 value of 1.80×10⁻⁷, and bound weakly to both CD4+ and CD8+ T cells, with EC50 values of 1.67E-07M and 1.80E-07M, respectively.

The Binding of PSMAxCD28 bispecific antibodies to the surface of cell lines expressing PSMA was tested by flow cytometry. C4-2 is a prostate cancer cell line derived from LNCaP (androgen sensitive human prostate adenocarcinoma cells derived from lymph node metastasis; see Wu et al., Int. J. Cancer, 57:406-412 (1994)) cells. Both BSPSMA/CD28-001 and BSPSMA/CD28-003 bound to C4-2 cells (see Liu et al., 2004, Prostate, 60:98-108) with EC50 values of 3.87×10⁻⁹ M and 1.50×10⁻⁹ M, respectively. 22RV1 is an epithelial prostate carcinoma cell line (see In Vitro Cell Dev. Biol. Anim., 1999, 35(7):403-409) Both BSPSMA/CD28-001 and BSPSMA/CD28-003 bound to 22RV1 cells with EC50 values of 3.05×10⁻⁹ M and 6.33×10⁻⁰⁹ M, respectively.

### Example 4: Primary and Engineered Bioassay for PSMAxCD28 Bispecific Antibodies

T-cell activation is achieved by stimulating T-cell receptors (TCR) that recognize specific peptides presented by major histocompatibility complex class I or II (MHCI or MHCII) proteins on antigen-presenting cells (APC) (Goldrath et al., Selecting and maintaining a diverse T-cell repertoire, Nature 402, 255-262 (1999)). An activated TCR in turn initiates a cascade of signaling events, which can be monitored by reporter genes, driven by various transcription factors such as activator-protein 1 (AP-1), Nuclear Factor of Activated T-cells (NFAT) or Nuclear factor kappa-light-chain-enhancer of activated B cells (NFκB). The T-cell response is then further refined via engagement of co-receptors expressed either constitutively or inducibly on T-cells such as CD28, CTLA-4 (Cytotoxic T-Lymphocyte-Associated Protein 4), PD-1 (Programmed Cell Death Protein 1), LAG-3 (Lymphocyte-Activation Gene 3) or other molecules (Sharpe et al., The B7—CD28 Superfamily, Nat. Rev. Immunol., 2(2): 116-26 (2002)). The co-stimulatory molecule, CD28, is activated by its endogenous ligands CD80 or CD86 expressed on APCs. CD28 potentiates cellular signals such as pathways controlled by the NFκB transcription factor after TCR activation. The CD28 co-signal is important for effective T-cell activation such as T cell differentiation, proliferation, cytokine release and cell-death (Smeets et al., NFκB activation induced by T cell receptor/CD28 costimulation is mediated by protein kinase C-θ, PNAS, 97(7):3394-3399 (2012).

In order to identify antibodies that enhance T cell activity in the presence of both primary stimulation and PSMA target expression, anti-PSMAxCD28 bispecific antibodies were characterized in an engineered reporter bioassay and cell-based assays using human primary T-cells. The assays evaluate the anti-PSMA/CD28 bispecific antibody's behavior in the presence and absence of primary stimulation and in the presence and absence of target expression. The assays were conducted to identify anti-PSMAxCD28 bispecific antibodies that enhance T cell activity in the presence of primary stimulation and target expression. Accordingly, the assays evaluated bispecific antibodies' behavior in the presence and absence of primary stimulation and target expression.

A Jurkat derived T-cell clone, JRT3.T3.5 (ATCC, #TIB-153) was transduced with an NFκB luciferase reporter construct (NFκB-Luc, SA Biosciences/Qiagen, Cat. #CLS-013L). After the isolation of a puromycin resistant clone (JRT3.T3.5/NFκB-Luc Clone 1C2), cells were further engineered to express full-length human TCR alpha (1G4A - amino acids M1 to S274) and TCR beta subunit (1G4B—amino acids M1 to G311) (Robbins et al., Single and Dual Amino Acid Substitutions in TCR CDRs Can Enhance Antigen-Specific T Cell Functions, J. Immunol. 180(9): 6116-31(2008)). After isolating a single clone (J.RT3-T3.5/NFκB-Luc/1G4AB Clone 1D2), cells were further engineered to express full-length human CD8 alpha (hCD8a—amino acids M1 to V235 of accession #NP_001139345) and human CD8 beta subunit (hCD8b—amino acids M1 to K210 of accession #P10966). A single clone was generated again (J.RT3-T3.5/NFκB-Luc/1G4AB/hCD8ab Clone 1 D5) and further transduced with full-length human CD28 (hCD28—amino acids M1 to S220 accession #P10747). Cells were sorted for high CD28 expression and maintained in RPMI+20% FBS+penicillin/streptomycin/glutamine (P/S/G)+NEAA+NaPyr+1 μg/mL puromycin+500 μg/mL G418+250 μg/mL hygromycin+10 μg/mL blasticidin. For faster growth, the engineered reporter T-cells were kept in cell culture media without antibiotics and used for cell-based luciferase experiments as engineered reporter T-cells. The reagents information is as follows: RPMI 1640, Irvine Scientific, Cat. #9160; FBS, Seradigm, Cat. #1500-50; Penicillin/Streptomycin/Glutamine 100× (P/S/G), Thermo Fisher Scientific, Cat. #10378-016; Non-Essential Amino-Acids (NEAA), Irvine Scientific, Cat. #9304; Sodium Pyruvate (NaPyr), Millipore, Cat. #TMS-005-C; puromycin, Sigma, Cat. #P8833; Geneticin (G418), Thermo Fisher Scientific, Cat. #11811-098; hygromycin; blasticidin.

A stable HEK293 cell line (ATCC, #CRL-1573) expressing human CD20 (amino acids M1 to P297 of accession number NP_068769.2) was transduced with human PSMA (amino acids M1 to A750 of accession number Q04609). Human PSMA positive cells were isolated by fluorescence-activated cell sorting (FACS) and the resulting cell line, HEK293/CD20/hPSMA high sorted was maintained in DMEM+10%+P/S/G+NEAA supplemented with 500 μg/mL G418.

In this experiment, engineered reporter T-cells are stimulated via two bispecific antibodies. The first stimulation is delivered by a T-cell activating bispecific antibody, anti-CD3xCD20 hIgG4, (see WO14/047231) targeting CD3 molecules on engineered reporter T-cells and CD20 on HEK293 cells. Here, the first stimulation bypasses the need of activation of TCRs by their natural ligands, which are specific peptides displayed on MHC molecules. The second stimulation is driven by the CD28 bispecific antibody. This antibody mimics the CD28 activation on T-cells by its ligands, CD80/CD86, expressed on APCs. Here, the antibody interacts with CD28 on T-cells and PSMA on engineered HEK293 cells and drives the activation of CD28 on engineered reporter T-cells. The simultaneous TCR and CD28 activation leads to enhanced transcriptional activity of NFκB, which in turn induces the production of the reporter gene, luciferase.

RPMI1640 supplemented with 10% FBS and P/S/G was used as the assay medium to prepare cell suspensions and antibody dilutions for screening of the anti-PSMA x CD28 bispecific antibodies. A day prior to screening, engineered reporter T-cells were cultured to 1×10⁶ cells/mL in cell culture media. Three fold (1:3) serially diluted anti-PSMA x CD28 bispecific antibodies and controls were tested in the presence of constant 50 pM anti-CD20 x CD3 or an hIgG4 isotype control. The 10-point dilution ranged between 15 pM to 100 nM with the final dilution containing no anti-PSMA x CD28 antibodies. Reagents were added in following order: 1) serially diluted antibodies were added to 96 well white flat bottom plates into corresponding wells; 2) A fixed concentration of 50 pM anti-CD20 x CD3 or hIgG4 isotype control was added to each well; 3) APCs re-suspended to 433 10⁵ cells/mL were added to plates with a final concentration 1×10⁴ cells/well; 4) Overnight cultured reporter T-cells were re-suspended at 2×10⁶/mL and added to plates with a final concentration 5×10⁴ cells/well. Plates were incubated for 4-6 hours at 37° C./5% CO₂, before the addition of 100 μL ONE-Glo™ (Promega, Cat. #E6051) reagent to lyse cells and detect luciferase activity. The emitted light was captured in relative light units (RLU) on a multilabel plate reader Envision (PerkinElmer, Model 2104). All serial dilutions were tested in duplicate.

The EC₅₀ values of the antibodies were determined by fitting the data to a four-parameter logistic equation over a 10-point dose-response curve using GraphPad Prism™ software. Fold induction was calculated using the following equation:

$$Fold induction = \frac{Mean RLU values of antibody [ 100 nM ]}{Mean RLU values of antibody [ 0 nM ]}$$

EC₅₀ and fold induction values are summarized in Tables 6 and 7 for engineered reporter T-cells co-incubated with HEK293/hCD20 or HEK293/hCD20/hPSMA cells in addition to either 50 pM constant hIgG4 isotype control or anti-CD3 x CD20 bispecific antibody (T-cell stimulating bispecific antibody).

TABLE 7

| | | | HEK293/ | |
|---|---|---|---|---|
| | HEK293/hCD20 | | hCD20/hPSMA | |
| | | Fold | | Fold |
| Antibodies | EC₅₀ [M] | induction | EC₅₀ [M] | induction |

Luciferase Activity in Engineered Reporter
T-Cells in Absence of TCR Stimulation

| | | | | |
|---|---|---|---|---|
| BSPSMA/CD28-001 | — | 0.81 | — | 1.04 |
| BSPSMA/CD28-002 | — | 0.87 | — | 0.89 |
| BSPSMA/CD28-003 | — | 0.94 | — | 0.98 |
| mAb14226P2 (CD28) | 5.11 E−09 | 4.76 | 4.90 E−09 | 3.59 |
| mAb14193P2 (CD28) | — | 0.86 | — | 0.89 |
| mAb14216P2 (CD28) | n/c | 2.80 | n/c | 3.95 |
| one-arm | — | 0.83 | — | 0.88 |
| mAb14226P2 | | | | |
| one-arm | — | 0.88 | — | 0.86 |
| mAb14193P2 | | | | |
| one-arm | — | 0.89 | — | 0.91 |
| mAb14216P2 | | | | |

Luciferase Activity in Engineered Reporter
T-cells in Presence of TCR Stimulation

| | | | | |
|---|---|---|---|---|
| BSPSMA/CD28-001 | — | 1.00 | 1.72 E−10 | 16.15 |
| BSPSMA/CD28-002 | — | 0.92 | 4.57 E−10 | 13.61 |
| BSPSMA/CD28-003 | — | 0.95 | 2.78 E−10 | 24.47 |
| mAb14226P2 (CD28) | 3.59 E−09 | 3.16 | 3.27 E−09 | 3.03 |
| mAb14193P2 (CD28) | 2.23 E−08 | 1.27 | — | 1.37 |
| mAb14216P2 (CD28) | n/c | 3.26 | n/c | 3.15 |
| one-arm | — | 0.99 | — | 0.95 |
| mAb14226P2 | | | | |
| one-arm | 2.24 E−08 | 1.10 | — | 1.15 |
| mAb14193P2 | | | | |
| one-arm | — | 0.96 | — | 0.94 |
| mAb14216P2 | | | | |

When T-cells and APCs are treated with 50 pM hIgG4 isotype control, none of the CD28 bispecific antibodies showed an increase in luciferase activity in the absence of TCR stimulation, irrespective of the APC line used in the assay. A slight luciferase activation was observed with one of the parental CD28 antibodies (mAb14226P2) on HEK293/hCD20 cells (4.76×) and HEK293/hCD20/hSPMA cells (3.59×) shown in Table 7A. mAb14226P2, mAb14193P2 and mAb14216P2 correspond, respectively, to the parental anti-CD28 antibodies of BSPSMA/CD28-001, BSPSMA/CD28-002, and BSPSMA/CD28-003.

In contrast, if cells were treated with 50 pM anti-CD3 x CD20 bispecific antibody, all three anti-PSMA x CD28 bispecific antibodies BSPSMA/CD28-001, BSPSMA/CD28-002, and BSPSMA/CD28-003 strongly induced luciferase activity when co-incubated with APCs expressing hPSMA on the surface. Very low to no activation was observed with their one-armed parental CD28 controls (one arm of mAb14226P2, mAb14193P2, and mAb14216P2) irrespective of the APC line. A slight luciferase activation was observed for all three parental CD28 antibodies (mAb14226P2, mAb14193P2, and mAb14216P2).

### Example 5: A Phase 1/2 Study of a Bispecific Anti-PSMA x Anti-CD28 Antibody Administered in Combination with an Anti-PD-1 Antibody in Patients with Metastatic Castration-Resistant Prostate Cancer

This is an open-label, phase 1/2, first-in-human study evaluating safety, tolerability, pharmacokinetics (PK), and anti-tumor activity of mAb1 (REGN5678) alone and in combination with cemiplimab in treatment-experienced metatstatic castration-resistant prostate cancer (mCRPC).

### Study Objectives The primary objectives of the study are:

To evaluate safety, tolerability and pharmacokinetics (PK) of mAb1 alone and in combination with cemiplimab (dose escalation)
To assess efficacy, as measured by objective response rate (ORR) per modified Prostate Cancer Working Group 3 (PCWG3) criteria, of mAb1 in combination with cemiplimab (dose expansion)

The secondary objectives of the study are:
To assess efficacy, as measured by ORR per modified PCWG3 criteria, of mAb1 in combination with cemiplimab (dose escalation)
To characterize the safety profile in each expansion cohort (dose expansion)
To characterize the PK of mAb1 in combination with cemiplimab (dose expansion)
To assess efficacy of mAb1 in combination with cemiplimab as measured by additional criteria (dose escalation and dose expansion)
To assess immunogenicity of mAb1 in combination with cemiplimab (dose escalation and dose expansion)

All of the primary, secondary and exploratory objectives of the study will apply to each cohort in the study including those who receive sarilumab and those who do not receive sarilumab.

### Study Design

This is an open-label, phase 1/2, first-in-human study evaluating safety, tolerability, PK, and anti-tumor activity of mAb1 (anti-PSMAxCD28) alone and in combination with cemiplimab (anti-PD-1) in treatment-experienced metastatic castration-resistant prostate cancer (mCRPC). There are 2 parts of the study: Dose escalation; and Dose expansion.

Dose escalation: During dose escalation, patients will receive a 3-week monotherapy lead-in of mAb1 at the assigned dose level (DL) intravenously (IV) one weekly (QW) followed by combination therapy of mAb1 at the assigned DL IV QW and cemiplimab 350 mg IV one every three weeks (Q3W). Once a minimum pharmacologically active dose level is identified, the dosing interval for mAb1 will be extended from QW to Q3W for subsequent dose escalation. Once a maximum tolerated dose (MTD)/presumptive recommended phase 2 dose(s) (presumptive RP2D) of mAb1 IV is identified, subcutaneous (SC) dosing of mAb1 may be explored. The tolerability of concomitant dosing of mAb1 and cemiplimab (without a monotherapy lead-in of mAb1) with or without sarilumab 350 mg IV Q3W×4 doses may be investigated at the MTD/presumptive RP2D following initiation of the expansion phase (*cohorts).

The use of sarilumab prophylaxis at 350 mg IV Q3W×4 doses will be explored during dose escalation in combination with REGN5678 and cemiplimab. Dose level (DL) 7 (100 mg REGN5678 IV QW) has cleared DLT evaluation and deemed tolerable, and, therefore, was selected for the initial cohort with sarilumab. Based upon safety and tolerability of this initial cohort, dose escalation may continue with sarilumab prophylaxis. Eligibility criteria was changed specifically for patients in the sarilumab cohort(s), including increasing the absolute neutrophil count study inclusion threshold to ≥1.5×109/L, and excluding patients with active or prior tuberculosis (TB), prior opportunistic infections, bowel perforation, severe diverticulitis, or inflammatory bowel disease. Based upon ongoing evaluation of cumulative safety and tolerability data in all cohorts, escalation above 100 mg (DL7) may proceed with and/or without sarilumab prophylaxis. The addition of IL-6R blockade with sarilumab may mitigate immune-mediated adverse events (imAEs) that arise from treatment with REGN5678 in combination with cemiplimab while preserving anti-tumor activity.

Dose Expansion: During dose expansion, patients will receive a 3-week monotherapy lead-in of mAb1 followed by combination therapy of mAb1 at the MTD/presumptive RP2D(s) IV and cemiplimab 350 mg IV Q3W. A dose expansion cohort utilizing SC dosing of mAb1 at the MTD/presumptive RP2D may also be investigated.

Dose expansion cohort(s) will be enrolled after identification of the mAb1 MTD in combination with cemiplimab and/or presumptive RP2D. Safety evaluations will be conducted at each study drug dosing visit. Radiographic response assessment will be performed every 6 weeks from cycle 1 (C1 D42/C2D1) up to cycle 4 (including patients who receive the initial 3-week monotherapy lead-in of mAb1) and every 12 weeks thereafter.

### Study Duration

The total duration of study participation for each patient will vary based on the occurrence of 1 or more of the following: disease progression, intolerable adverse events (AEs), withdrawal of consent, or study withdrawal criterion is met.

For patients in dose escalation and expansion cohorts (with the exception of *cohorts in which there is no monotherapy lead-in period), the study consists of 4 periods: a screening period (up to 28 days), a 3-week monotherapy lead-in period of mAb1 (21 days), a combination treatment period consisting of a series of 6-week (42 day) cycles of mAb1 in combination with cemiplimab with or without sarilumab (variable duration until discontinuation), and an off-treatment follow-up period (90 days). For patients in *cohorts, the study consists of 3 periods: a screening period (up to 28 days), a combination treatment period consisting of a series of 6-week (42 day) cycles of mAb1 in combination with cemiplimab (variable duration until discontinuation), and an off-treatment follow-up period (90 days).

Dose escalation of mAb1 will proceed QW until identification of a pharmacologically active dose level (i.e., minimum pharmacologically active dose). Once the minimum pharmacologically active dose is identified, mAb1 dose escalation will proceed to the next dose level and will be administered Q3W.

TABLE 8
Planned Dose Escalation Cohorts and Enrollment

| Dose Level | mAb1 | | Cemiplimab | Initial Enrollment |
|---|---|---|---|---|
| DL1a | 0.03 | mg | 350 mg | 0-4 |
| DL1 | 0.1 | mg | 350 mg | 1-2 |
| DL2 | 0.3 | mg | 350 mg | 1-2 |
| DL3 | 1 | mg | 350 mg | 3-4 |
| DL4 | 3 | mg | 350 mg | 3-4 |
| DL4a | 6 | mg | 350 mg | 0-4 |
| DL5 | 10 | mg | 350 mg | 3-4 |
| DL5a | 20 | mg | 350 mg | 0-4 |
| DL6 | 30 | mg | 350 mg | 3-4 |
| DL6a | 60 | mg | 350 mg | 0-4 |
| DL7 | 100 | mg | 350 mg | 3-12 |
| DL7a.1 | 150 | mg | 350 mg | 0-12 |
| DL7a | 200 | mg | 350 mg | 3-12 |
| DL7a.2 | 250 | mg | 350 mg | 0-12 |
| DL8 | 300 | mg | 350 mg | 3-12 |
| DL8a.1 | 450 | mg | 350 mg | 0-12 |
| DL8a | 600 | mg | 350 mg | 3-12 |
| DL8a.2 | 750 | mg | 350 mg | 0-12 |
| DL9 | 900 | mg | 350 mg | 3-12 |

Cohorts marked “a.1” or “a.2” are intermediate cohorts that may be used if a smaller dose increase is desired.

### Study Population

Approximately 216 (approximately 108 patients during dose escalation, and approximately up to 3-4 expansion cohorts with a maximum of 27 patients each) patients will be enrolled. The study population includes men with treatment-experienced mCRPC. For inclusion in this study, patients must have received at least 2 lines of prior systemic therapy (in addition to androgen deprivation therapy [ADT]) approved for metastatic and/or castration-resistant disease, including a second-generation anti-androgen therapy (e.g., abiraterone, enzalutamide, apalutamide, or darolutamide).

Inclusion Criteria: A patient must meet the following criteria to be eligible for inclusion in the study:
1. Men ≥18 years of age
2. Histologically or cytologically confirmed adenocarcinoma of the prostate without pure small cell carcinoma.
3. Metastatic, castration-resistant prostate cancer (mCRPC) with PSA value at screening ≥4 ng/mL that has progressed within 6 months prior to screening according to 1 of the following:
   a. PSA progression as defined by a rising PSA level confirmed with an interval of ≥1 week between each assessment.
   b. Radiographic disease progression in soft tissue based on RECIST Version 1.1 criteria with or without PSA progression
   c. Radiographic disease progression in bone defined as the appearance of 2 or more new bone lesions on bone scan with or without PSA progression
4. Has received ≥2 lines prior systemic therapy approved in the metastatic and/or castration-resistant setting (in addition to androgen deprivation therapy [ADT]) including at least
   a. one second-generation anti-androgen therapy (e.g., abiraterone, enzalutamide, apalutamide, or darolutamide)

NOTE: a non-taxane based chemotherapy regimen given for metastatic prostate cancer with mixed histology is permissible and will be included when evaluating line of therapy
5. Able and willing to provide tumor tissue, either archival or newly obtained. NOTE: For dose escalation only, if archival or fresh tissue is not available, a pathology report that confirms diagnosis of prostate cancer may be submitted.
6. Have had either orchiectomy OR be on luteinizing hormone-releasing hormone (LHRH) agonist or antagonist therapy with serum testosterone <50 ng/dL AND agree to stay on LHRH agonist or antagonist therapy during the study
7. Eastern Cooperative Oncology Group (ECOG) performance status of 0 or 1
8. Adequate organ and bone marrow function documented by:
   a. Hemoglobin ≥8.5 g/dL
   b. Absolute neutrophil count ≥1.0×10⁹/L (for sarilumab cohorts only: ≥1.5×10⁹/L)
   c. Platelet count ≥100×10⁹/L
9. Serum creatinine ≥1.5×ULN or estimated glomerular filtration rate >50 mL/min/1.73 m 2 . A 24-hour urine creatinine collection may substitute for the calculated creatinine clearance to meet eligibility criteria
10. Adequate hepatic function:
   a. Total bilirubin ≤1.5×ULN (≤3×ULN if tumor liver involvement)
   b. AST ≤2.5×ULN (5×ULN if tumor liver involvement)
   c. ALT ≤2.5×ULN (≤5×ULN if tumor liver involvement)
   d. Alkaline Phosphatase (ALP) ≤2.5×ULN (≤5×ULN if tumor liver or bone involvement) NOTES:
   In patients with tumor liver involvement if levels of AST ≥3×ULN or ALT ≥3×ULN, and bilirubin levels ≥2×ULN will be excluded regardless of the above criteria
   Patients with Gilbert's syndrome do not need to meet total bilirubin requirements provided their total bilirubin is not greater than their historical level. Gilbert's syndrome must be documented appropriately as past medical history
11. Willing and able to comply with clinic visits and study-related procedures
12. Provide informed consent signed by study patient
13. Able to understand and complete study-related questionnaires

Exclusion Criteria: A patient who meets any of the following criteria will be excluded from the study:
1. Currently receiving treatment in another study
2. Has participated in a study of an investigational agent or an investigational device within 4 weeks of first dose of study therapy
3. Has received treatment with an approved systemic therapy (including sipuleucel-T) within 3 weeks of dosing or has not yet recovered (i.e., grade ≤1 or baseline) from any acute toxicities except for laboratory changes as described in inclusion criteria and as below:
   a. Patients with grade 2≤neuropathy
4. Has received radiation therapy or major surgery within 14 days of first administration of study drug or has not recovered (i.e., grade ≤1 or baseline) from AEs, except for laboratory changes as described in inclusion criteria and as below:
   a. Patients with grade neuropathy
5. Has received any previous systemic biologic therapy within 5 half-lives of first dose of study therapy
   Exception: Patients previously treated with bevacizumab, or other non-immunomodulatory antibodies with half-lives longer than 7 days are permitted after a discussion with the sponsor if at least 30 days have elapsed since last treatment.
6. Has received prior PSMA-targeting therapy
7. Dose Escalation: Has had prior anti-cancer immunotherapy (other than sipuleucel-T) within half-lives prior to study therapy. Examples of immune modulating agents include blockers of CTLA-4, 4-1 BB (CD137), or OX-40, therapeutic vaccines, anti-PD-1/PD-L1, phosphoinositide 3-kinase (P13K) delta inhibitors, or cytokine anticancer treatments. NOTE: Patients who have received prior investigational cell-based therapies (e.g., CAR-T cells) are excluded.
8. Dose Expansion: Has had prior anti-cancer immunotherapy. Examples of immune modulating agents include blockers of CTLA-4, 4-1BB (CD137), or OX-40, therapeutic vaccines, anti-PD-1/PD-L1, PI3Kdelta inhibitors, CAR-T cells, or cytokine anticancer treatments. NOTE: Prior treatment with sipuleucel-T is permitted
9. Patients who have not recovered (i.e., grade ≤1 or baseline) from immune-mediated AEs 3 months prior to initiation of study drug therapy except for endocrinopathies adequately managed with hormone replacement
10. Patients who have permanently discontinued anti-cancer immune modulating therapies due to immune-related AEs
11. Another malignancy that is progressing or requires active treatment, except:
   a. Non-melanoma skin cancer that has undergone potentially curative therapy
   b. Any tumor that has been deemed to be effectively treated with definitive local control (with or without continued adjuvant hormonal therapy)
12. Any condition that requires ongoing/continuous corticosteroid therapy (>10 mg prednisone/day or anti-inflammatory equivalent) within 1 week prior to the first dose of study therapy. Patients who require a brief course of steroids (up to 2 days in the week before enrollment) or physiologic replacement are not excluded
13. Ongoing or recent (within 5 years) evidence of significant autoimmune disease that required treatment with systemic immunosuppressive treatments. The following are not exclusionary: vitiligo, childhood asthma that has resolved, endocrinopathies (such as hypothyroidism or type 1 diabetes) that require only hormone replacement, or psoriasis that does not require systemic treatment
14. History of CNS metastases, including previously treated metastases
15. Encephalitis, meningitis, neurodegenerative disease (with the exception of mild dementia that does not interfere with activities of daily living [ADLs]) or uncontrolled seizures in the year prior to first dose of study therapy
16. Known history of, or any evidence of interstitial lung disease, or active, non-infectious pneumonitis (past 5 years). A history of radiation pneumonitis in the radiation field is permitted
17. Uncontrolled infection with human immunodeficiency virus (HIV), hepatitis B or hepatitis C infection; or diagnosis of immunodeficiency

### NOTES:

Patients will be tested for hepatitis C virus (HCV) and hepatitis B virus (HBV) at screening.
Patients with known HIV infection who have controlled infection (undetectable viral load (HIV RNA polymerase chain reaction [PCR]) and CD4 count above 350 either spontaneously or on a stable antiviral regimen) are permitted. For patients with controlled HIV infection, monitoring will be performed per local standards.
Patients with hepatitis B (HepBsAg+) who have controlled infection (serum hepatitis B virus DNA PCR that is below the limit of detection AND receiving antiviral therapy for hepatitis B) are permitted. Patients with controlled infections must undergo periodic monitoring of HBV DNA. Patients must remain on antiviral therapy for at least 6 months beyond the last dose of investigational study drug.
Patients who are hepatitis C antibody positive (HCV Ab+) who have controlled infection (undetectable HCV RNA by PCR either spontaneously or in response to a successful prior course of anti-HCV therapy) are permitted.
18. Any infection requiring hospitalization or treatment with IV anti-infectives within 2 weeks of first dose of study therapy
19. Receipt of a live vaccine within 4 weeks of planned start of study medication.
20. Prior allogeneic stem cell transplantation or recipients of organ transplants at any time, or autologous stem cell transplantation within 12 weeks of the start of study treatment
21. Has known allergy or hypersensitivity to cemiplimab or components of study drugs.
22. Known psychiatric or substance abuse disorders that would interfere with participation with the requirements of the study
23. Any medical condition, co-morbidity, physical examination finding, metabolic dysfunction, or clinical laboratory abnormality that, in the opinion of the investigator, renders the patient unsuitable for participation in a clinical trial due to high safety risks and/or potential to affect interpretation of results of the study including, but not limited to, significant cardiovascular disease (e.g., New York Heart Association Class III or IV cardiac disease, myocardial infarction within the previous 6 months, unstable arrhythmias or unstable angina) and/or significant pulmonary disease (e.g., obstructive pulmonary disease and history of symptomatic bronchospasm).
24. Sarilumab cohorts only: exclusion criteria related to tuberculosis (TB)
   a. Active TB or a history of incompletely treated TB
   b. QuantiFERON-positive patients (no active disease) are excluded from the study unless there is a history of prior documented completed chemoprophylaxis for latent tuberculosis infection (LTBI) (eg, acceptable treatments would be 9 months of isoniazid 300 mg by mouth daily or equivalent proven regimen per local guidelines) or treatment of active tuberculosis infection (TBI) and has obtained consultation with a specialist to rule out active disease
25. Sarilumab cohorts only: patients with a history of invasive opportunistic infections, including but not limited to histoplasmosis, coccidioidomycosis, Pneumocystic jirovecii, or aspergillosis, or John Cunningham virus (progressive multifocal leukoencephalopathy).
26. Sarilumab cohorts only: History of bowel perforation, severe diverticulitis, or inflammatory bowel disease

### Study Treatments

mAb1 at the assigned dose level will be administered QW or Q3W either by IV infusion over 30 minutes to 2 hours or by SC injection. Cemiplimab 350 mg will be administered by IV infusion over 30 minutes Q3W. Sarilumab 350 mg Q3W will be administered by IV infusion over 60 minutes for four doses (a total of 12 weeks) starting with the initial dose of REGN5678 in combination with cemiplimab. When both mAb1 and cemiplimab are administered on the same day, mAb1 will be administered first. For cohorts receiving sarilumab IV, it should be administered prior to mAb1 and cemiplimab. In select patients at select sites, 18 F-DCFPyL will be administered for experimental PSMA PET/CT imaging procedures.

### Study Endpoints

The study's primary endpoints are:

### Dose Escalation:

Safety, as measured by the incidence and severity of treatment-emergent adverse events (TEAEs)/adverse events of special interest (AESIs)/serious adverse events (SAEs) and grade ≥3 laboratory abnormalities during the treatment period and up to 90 days after the last dose of mAb1 and cemiplimab or until the start of new therapy for treatment of the patient's tumor, whichever occurs first.
Tolerability, as measured by the incidence of dose-limiting toxicities (DLTs) from the first dose through the end of the DLT observation period for mAb1 alone and in combination with cemiplimab
mAb1 concentrations in serum over time, dosed either alone or in combination with cemiplimab

### Dose Expansion:

ORR per modified PCWG3 criteria, defined as the percentage of patients who have achieved response per modified PCWG3 criteria based on:
   ≥50% decline of prostate specific antigen (PSA) from baseline and from start of combination therapy, confirmed by a second PSA test ≥4 weeks later, AND/OR
   Confirmed radiographic response of complete response (CR) or partial response (PR)
      All of the primary endpoints will apply to each cohort in the study including those who receive sarilumab and those who do not receive sarilumab.

The study's secondary endpoints are:

### Dose Escalation:

ORR, defined as the percentage of patients who have achieved response per modified PCWG3 criteria based on:
   ≥50% decline of PSA from baseline and from start of combination therapy, confirmed by a second PSA test ≥4 weeks later, AND/OR
   Confirmed radiographic response of CR or PR

### Dose Expansion:

Safety, as measured by the incidence and severity of treatment-emergent adverse events (AEs)/AESIs/SAEs and grade laboratory abnormalities during the treatment period and up to 90 days after the last dose of mAb1 in combination with cemiplimab or until the start of new therapy for treatment of the patient's tumor, whichever occurs first
mAb1 concentrations in serum over time when dosed in combination with cemiplimab

### Dose Escalation and Dose Expansion:

ORR based upon PSA response, defined as the percentage of patients who have achieved ≥50% decline of PSA from baseline and from start of combination therapy, confirmed by a second PSA test ≥4 weeks later
Percentage of patients with ≥90% decline of PSA from baseline and from start of combination therapy, confirmed by a second PSA test ≥4 weeks later
Percentage of patients who have achieved conversion of circulating tumor cell (CTC) count from baseline and from start of combination therapy of ≥5 cells/7.5 mL to <5 cells/7.5 mL
Presence or absence of antibodies against mAb1 and Cemiplimab
   All of the secondary endpoints will apply to each cohort in the study including those who receive sarilumab and those who do not receive sarilumab.

The study's exploratory endpoints are:
Percent change in PSA, defined as the largest percent change in PSA decline from baseline and from start of combination therapy
Percent change in PSA during 3-week monotherapy of mAb1, defined as the largest percent change in PSA decline from baseline during monotherapy lead-in period of mAb1
Radiographic progression-free survival (rPFS) (via modified PCWG3 and iRECIST), defined as the time from first study treatment administration to first radiographic progression or death due to any cause. Radiographic progression includes progression per modified PCWG3 of soft tissue lesions and bone lesions. In the absence of radiographic progression or death before the analysis cut-off date or the date of initiation of a further anticancer treatment, the rPFS will be censored at the date of the last valid radiographic response assessment not showing radiographic progression performed prior to the analysis cut-off date or initiation of a further anticancer treatment, whichever is earlier
PSA progression free survival (PFS), defined as the time from first study treatment administration and from start of combination therapy to first PSA progression or death due to any cause. PSA progression is defined as:
   After decline from baseline: PSA increase that is ≥25% and ≥2 ng/mL above the nadir, and which is confirmed by a second PSA test ≥4 weeks later;
   No decline from baseline: PSA progression that is a ≥25% increase and ≥2 ng/mL increase from baseline beyond 12 weeks
      In the absence of PSA progression or death before the analysis cut-off date or the date of initiation of a further anticancer treatment, the PSA PFS will be censored at the date of the last valid PSA test not showing PSA progression performed prior to the analysis cut-off date or initiation of a further anticancer treatment, whichever is earlier
ORR based upon radiographic response, defined as the percentage of patients who have achieved radiographic response of CR or PR (via modified PCWG3 and iRECIST)
Time to response based upon radiographic response (via modified PCWG3 and iRECIST), defined as the time from first study treatment administration to first radiographic response of CR or PR for patients with confirmed radiographic response of CR or PR
Time to response based upon PSA response, defined as the time from first study treatment administration to first PSA response for patients with confirmed PSA response
DOR based upon radiographic response (rDOR) (via modified PCWG3 and iRECIST), defined as time from first radiographic response of CR or PR to first radiographic progression or death for patients with confirmed radiographic response of CR or PR. The same censoring rule as rPFS will be used
DOR based upon PSA response, defined as the time from the first PSA response to first PSA progression for patients with confirmed PSA response. The same censoring rule as PSA PFS will be used
Time to progression based upon radiographic progression (via modified PCWG3 and iRECIST), defined as the time from first study treatment administration to first radiographic progression. In the absence of radiographic progression before the analysis cut-off date or the date of initiation of a further anticancer treatment, the time to progression will be censored at the date of the last valid radiographic response assessment not showing radiographic progression performed prior to the analysis cut-off date or initiation of a further anticancer treatment, whichever is earlier
Time to progression based upon PSA progression, defined as the time from first study treatment administration or start of combination therapy to first PSA progression. In the absence of PSA progression before the analysis cut-off date or the date of initiation of a further anticancer treatment, the time to progression will be censored at the date of the last valid PSA test not showing PSA progression performed prior to the analysis cut-off date or initiation of a further anticancer treatment, whichever is earlier
Disease control rate (DCR) (via modified PCWG3 and iRECIST), defined as the percentage of patients who have achieved radiographic response of CR, PR, SD, or Non-CR/Non-PD
OS, defined as the time from first study treatment administration to death due to any cause. In the absence of a survival event, OS will be censored at the last date that patient is known to be alive
Changes in CTC count (cells/7.5 mL) in quantifiable samples
Change in serum cytokines and other biomarkers of inflammation
Time to Pain Progression (TTPP) as Assessed by BPI-SF Item 3 (“Worst Pain in 24 Hours”) and Opiate Analgesic Use
Change from baseline in pain severity and pain interference as measured by the BPI-SF
Change from baseline in GHS/QoL as measured by the EORTC QLQ-C30 GHS/QoL scale score
Change from baseline in physical functioning as measured by the EORTC QLQ-C30 physical functioning scale score
Change from baseline in urinary symptoms as measured by the EORTC QLQ-PR25 urinary symptom scale score
Change from baseline in Patient Global Impression of Severity Score (PGIS)
Change from baseline in Patient Global Impression of Change Score (PGIC)
Correlation of levels of baseline tumor tissue biomarkers of mAb1 and cemiplimab target pathways (e.g., PD-L1, PSMA, and CD28) with therapeutic activity in available tissue biopsies
Association of tumor gene variants, including HDR mutations and TMB with ORR
Change from baseline in PSMA and FDG PET/CT tumor signal intensity after treatment initiation
Correlation of baseline PSMA PET/CT tumor positivity with clinical activity of mAb1 and cemiplimab
   All of the exploratory endpoints will apply to each cohort in the study including those who receive sarilumab and those who do not receive sarilumab.

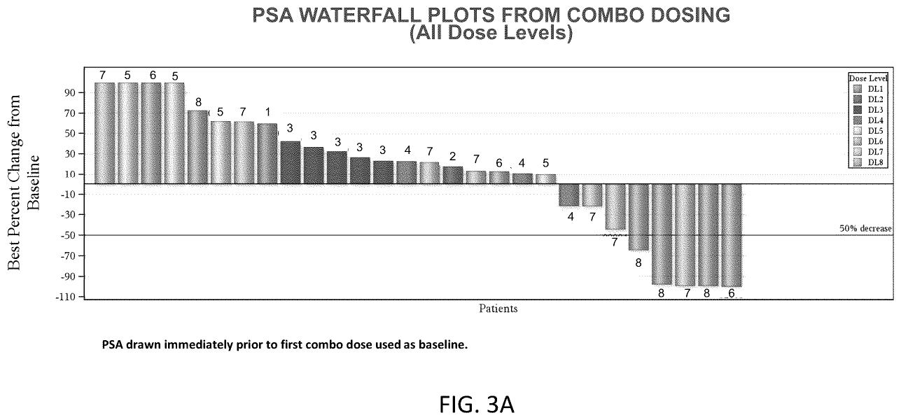

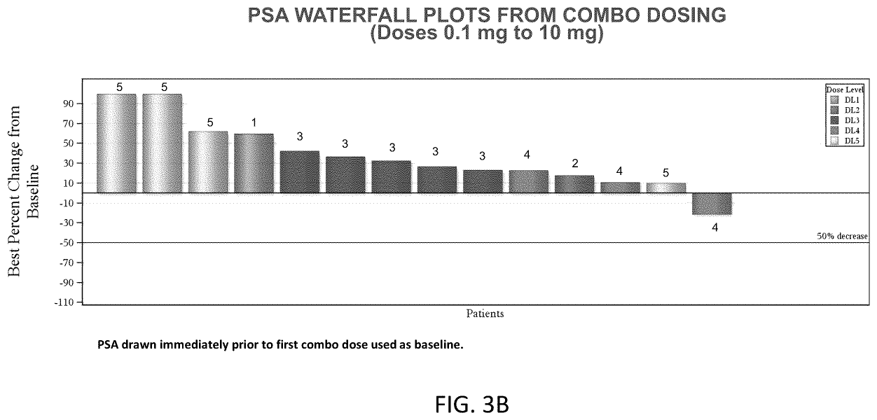

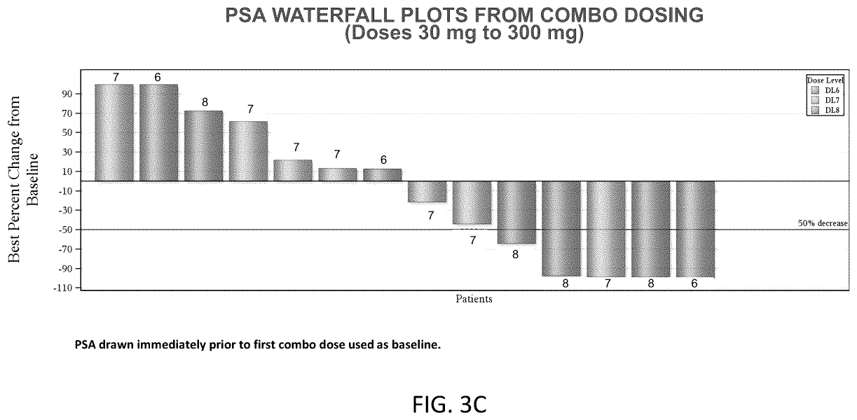

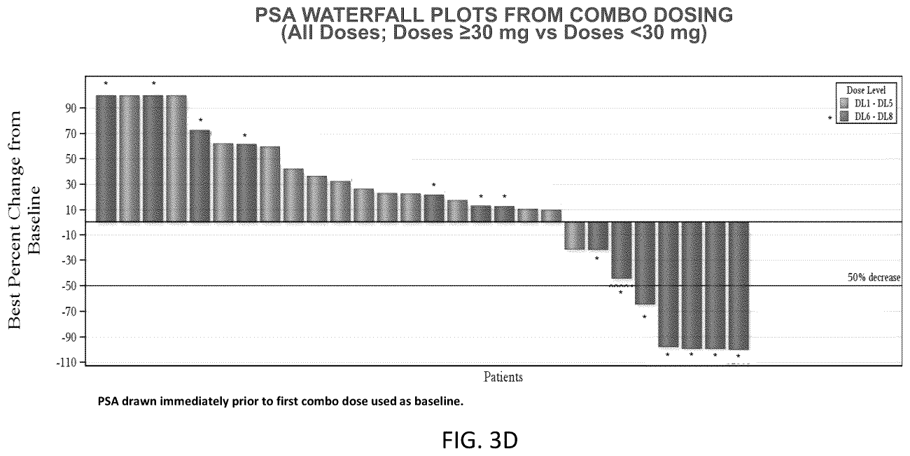

Results—anti-tumor activity was observed in patients treated with the combination of mAb1 and cemiplimab with a manageable safety profile. Prostate cancer (e.g., metastatic castration-resistant prostate cancer) patients treated with from 30 mg to 300 mg (to date) of mAb1 weekly, and cemiplimab (350 mg every three weeks) showed anti-tumor activity measured by decreases in prostate specific antigen (PSA) levels (e.g., greater than 50% decrease) and/or RESIST responses. Results are shown in FIGS. 3A, 3B, 3C and 3D, and in Table 9, below.

TABLE 9
Summary of Anti-Tumor Activity and Observed AEs at Doses up to 300 mg mAb1

| | | | | | Grade 3 |
|---|---|---|---|---|---|
| | | | | | Treatment |
| | | | Total | Anti-Tumor | Related |
| Dose Level (DL) | mAb1 Dose | | Enrollment | Activity (%) | Neurotoxicity (%) |
| DL1 | 0.1 | mg | 1 | 0 | 0 |
| DL2 | 0.3 | mg | 1 | 0 | 0 |
| DL3 | 1 | mg | 7 | 0 | 0 |
| DL4 | 3 | mg | 4 | 0 | 0 |
| DL5 | 10 | mg | 4 | 0 | 0 |
| DL6 | 30 | mg | 4 | 1 (25%) | 0 |
| DL7 | 100 | mg | 8 | 3 (37.5%) | 2 (25%) |
| DL8 | 300 | mg | 4 | 3 (75%) | 1 (25%) |

| | |
|---|---|
| Total | 33 |

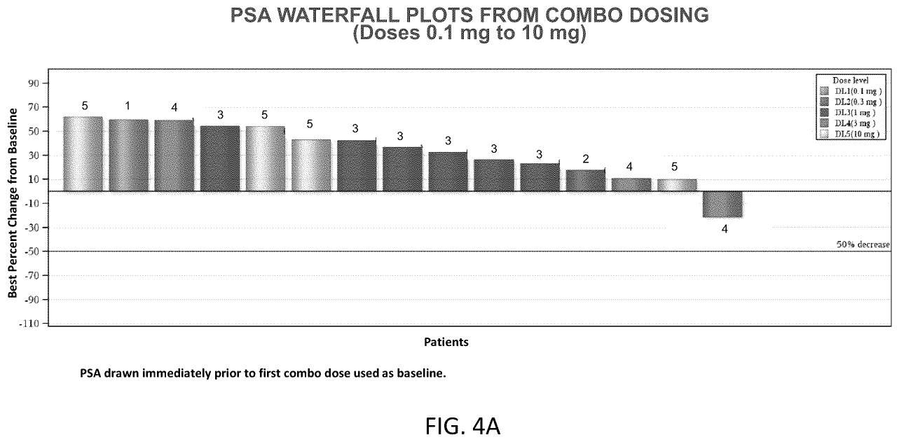

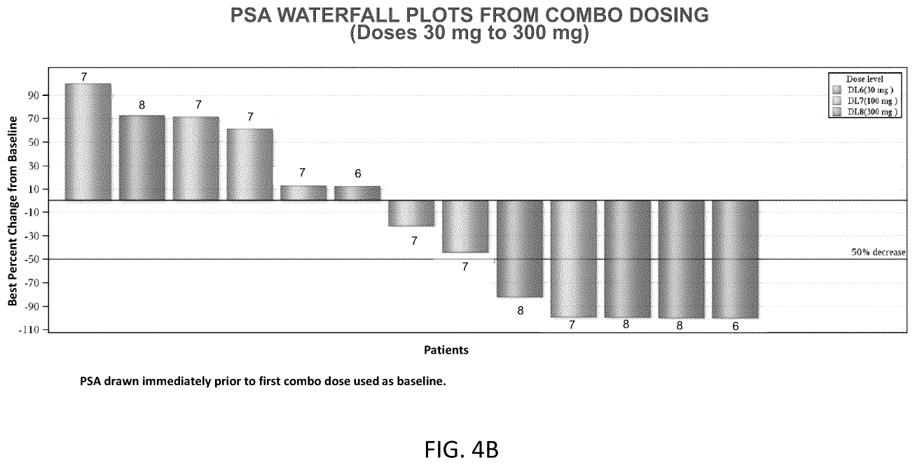

Updated anti-tumor data observed in 35 patients (17 at dose levels 1-5, and 18 at dose levels 6-8) is shown in FIGS. 4A and 4B. Notably, the patients receiving treatment at dose levels 1-5 showed no ≥Grade 3 immune-mediated adverse events (imAEs), and only 1 of 16 evaluable patients in dose levels 1-5 showed a decline is PSA levels. In contrast, patients receiving treatment at dose levels 6-8 showed signs of efficacy associated with imAEs. In dose level 6, 1 of 4 evaluable patients showed a response (a 100% decrease in PSA levels and a complete response in target lesions maintained for -12 months) along with a grade 3 imAE. In dose level 7, 3 of 8 evaluable patients showed a response (99%, 44% and 22% declines in PSA levels), two of which had a grade 3 imAE. In dose level 8, 3 of 4 evaluable patients showed a response (99%, 99% and 82% declines in PSA levels), one of which had an imAE resulting in death. Mitigation strategies to prevent imAEs, which appear to be associated with a therapeutic response, are under investigation, including blockade of IL-6R.

In each of the three patients receiving treatment at dose level 8 for which a response was observed, PSA levels continued to rise during the lead-in dosing of mAb1 until cemiplimab dosing was initiated. Advanced metastatic castration-resistant prostate cancer (mCRPC) shows about a 5% response rate to anti-PD-1 monotherapy, such that the observed rise in PSA levels until initiation of cemiplimab administration is evidence of a synergistic effect between mAb1 and cemiplimab in the mCRPC patients.

One patient receiving treatment at dose level 7 (99% decrease in PSA levels) showed pseudo-progression in the liver followed by a response, confirmed by a decrease in PSMA PET positive lesions. Notably, this same patient showed responses in tumor lesions with low PSMA PET signals that would not be expected to respond to Pluvicto™ (lutetium Lu 177 vipivotide tetraxetan), which comprises a PSMA-binding ligand bound to a DOTA chelator radiolabeled with lutetium-177, based on eligibility criteria.

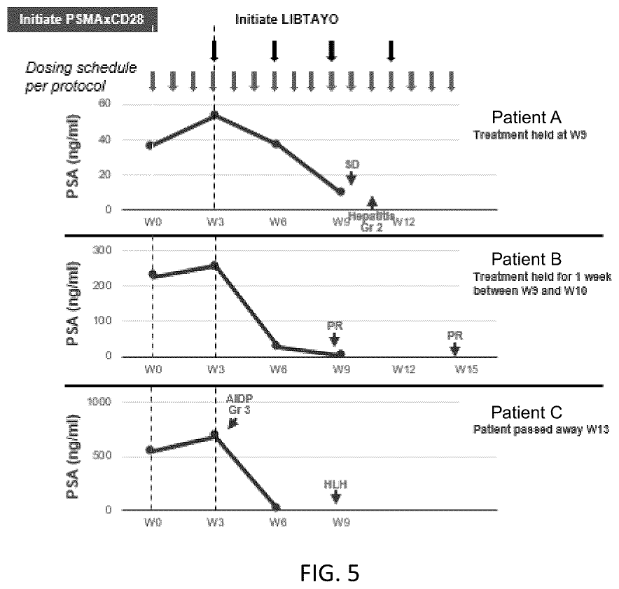

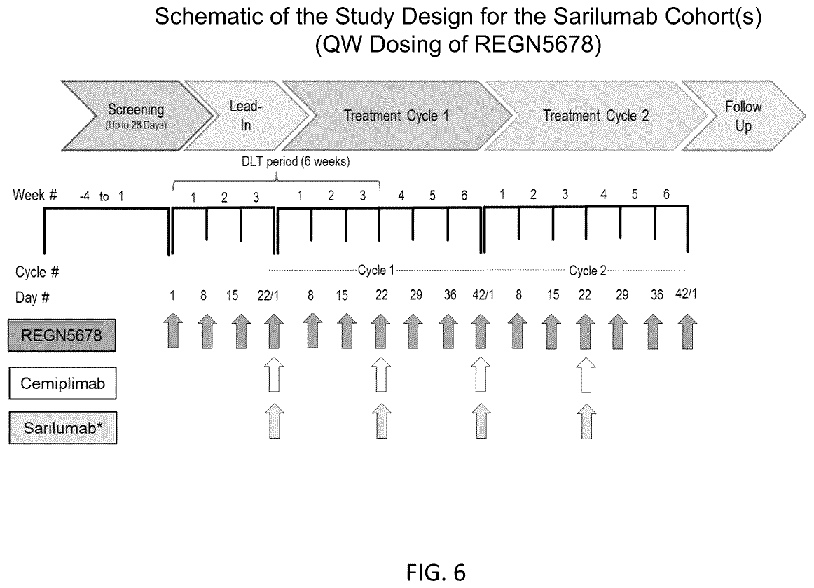

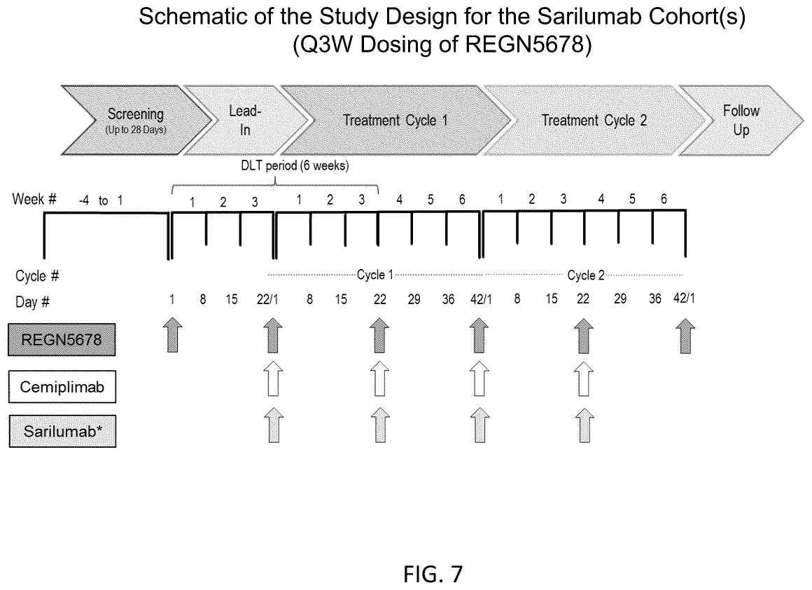

The initial results suggest minimal anti-tumor activity at lower doses, as predicted by preclinical models. However, anti-tumor activity amplified with cemiplimab initiation. At dose level 8, three of four patients showed profound PSA responses upon initiation of combination therapy (FIG. 5). In addition, one patient at dose level 6 experienced a response with PSA levels below the limit of detection, normalizing bone scan with negative PSMA PET scan and disappearance of soft tissue disease. However, this patient discontinued therapy due to a Grade 3 irAE of the skin that resolved with treatment. Grade 3 immune-related adverse events only occurred in certain patients with anti-tumor activity.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

TABLE 10
Sequences Excluded from
ST.26-Formatted Sequence Listing

| SEQ ID NO: | Sequence |
|---|---|
| 30 | AAS |
| 42 | AAS |

What is claimed is:

1. A method of treating a PSMA-expressing cancer in a subject in need thereof, comprising administering to the subject a combination of a bispecific antibody or antigen-binding fragment thereof comprising a first antigen-binding domain that specifically binds prostate specific membrane antigen (PSMA) on a target tumor cell, and a second antigen-binding domain that specifically binds human CD28 on a T cell, and an antibody or antigen-binding fragment thereof that specifically binds programmed death receptor-1 (PD-1), wherein the bispecific antibody is administered to the subject at a dose of at least 0.03 mg.

2. The method of claim 1, wherein the PSMA-expressing cancer is prostate cancer.

3. The method of claim 1 or 2, wherein the PSMA-expressing cancer is metastatic prostate cancer.

4. The method of any one of claims 1-3, wherein the PSMA-expressing cancer is castration-resistant prostate cancer.

5. The method of any one of claims 1-4, wherein the subject has received at least two prior therapies for metastatic and/or castration-resistant prostate cancer.

6. The method of claim 5, wherein the subject has received at least one anti-androgen therapy.

7. The method of claim 6, wherein the anti-androgen therapy is selected from abiraterone, enzalutamide, apalutamide, or darolutamide.

8. The method of any one of claims 1-7, wherein the subject has histologically or cytologically confirmed adenocarcinoma of the prostate without pure small cell carcinoma.

9. The method of any one of claims 1-8, wherein the subject has metastatic castration-resistant prostate cancer with a prostate specific antigen (PSA) value of ng/ml prior to treatment with the bispecific antibody.

10. The method of claim 9, wherein the subject's cancer has progressed within a six month period prior to treatment with the bispecific antibody, wherein cancer progression is determined by: (a) a rising PSA level confirmed with an interval of 1 week between each assessment; (b) radiographic disease progression in soft tissue with or without a rise in PSA; and/or (c) radiographic disease progression in bone with an appearance of two or more bone lesions on bone scan with or without a rise in PSA.

11. The method of any one of claims 1-10, wherein the subject has had an orchiectomy.

12. The method of any one of claims 1-10, wherein the subject is receiving luteinizing hormone-releasing hormone (LHRH) agonist or antagonist therapy, and has a serum testosterone level of <50 ng/ml prior to treatment with the bispecific antibody.

13. The method of any one of claims 1-12, wherein the first antigen-binding domain comprises:
(a) three heavy chain complementarity determining regions (HCDR1, HCDR2 and HCDR3) contained within a heavy chain variable region (HCVR) comprising the amino acid sequence of SEQ ID NO: 1; and
(b) three light chain complementarity determining regions (LCDR1, LCDR2 and LCDR3) contained within a light chain variable region (LCVR) comprising the amino acid sequence of SEQ ID NO: 9.

14. The method of claim 13, wherein the first antigen-binding domain comprises a HCDR1 comprising the amino acid sequence of SEQ ID NO: 2, a HCDR2 comprising the amino acid sequence of SEQ ID NO: 3, and a HCDR3 comprising the amino acid sequence of SEQ ID NO: 4.

15. The method of claim 13 or 14, wherein the first antigen-binding domain comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

16. The method of any one of claims 13-15, wherein the first antigen-binding domain comprises a HCVR comprising the amino acid sequence of SEQ ID NO: 1, and a LCVR comprising the amino acid sequence of SEQ ID NO: 9.

17. The method of any one of claims 1-16, wherein the second antigen-binding domain comprises:
(a) three heavy chain complementarity determining regions (HCDR1, HCDR2 and HCDR3) contained within a heavy chain variable region (HCVR) comprising the amino acid sequence of SEQ ID NO: 5; and
(b) three light chain complementarity determining regions (LCDR1, LCDR2 and LCDR3) contained within a light chain variable region (LCVR) comprising the amino acid sequence of SEQ ID NO: 9.

18. The method of claim 17, wherein the second antigen-binding domain comprises a HCDR1 comprising the amino acid sequence of SEQ ID NO: 6, a HCDR2 comprising the amino acid sequence of SEQ ID NO: 7, and a HCDR3 comprising the amino acid sequence of SEQ ID NO: 8.

19. The method of claim 17 or 18, wherein the second antigen-binding domain comprises a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

20. The method of any one of claims 17-19, wherein the second antigen-binding domain comprises a HCVR comprising the amino acid sequence of SEQ ID NO: 5, and a LCVR comprising the amino acid sequence of SEQ ID NO: 9.

21. The method of any one of claims 1-20, wherein the bispecific antibody comprises a human IgG heavy chain constant region.

22. The method of claim 21, wherein the human IgG heavy chain constant region is isotype IgG1.

23. The method of claim 21, wherein the human IgG heavy chain constant region is isotype IgG4.

24. The method of claim 22 or 23, wherein the bispecific antibody comprises a chimeric hinge that reduces Fcγ receptor binding relative to a wild-type hinge of the same isotype.

25. The method of any one of claims 21-24, wherein the first heavy chain or the second heavy chain, but not both, comprises a CH3 domain comprising a H435R (EU numbering) modification and a Y436F (EU numbering) modification.

26. The method of any one of claims 1-20, wherein the bispecific antibody comprises a first heavy chain comprising the amino acid sequence of SEQ ID NO: 13.

27. The method of any one of claims 1-20, wherein the bispecific antibody comprises a second heavy chain comprising the amino acid sequence of SEQ ID NO: 14.

28. The method of any one of claims 1-20, wherein the bispecific antibody comprises a first heavy chain comprising the amino acid sequence of SEQ ID NO: 13, a second heavy chain comprising the amino acid sequence of SEQ ID NO: 14, and a common light chain comprising the amino acid sequence of SEQ ID NO: 15.

29. The method of any one of claims 1-12, wherein the first antigen-binding domain comprises:
(a) three heavy chain complementarity determining regions (HCDR1, HCDR2 and HCDR3) contained within a heavy chain variable region (HCVR) comprising the amino acid sequence of SEQ ID NO: 16; and
(b) three light chain complementarity determining regions (LCDR1, LCDR2 and LCDR3) contained within a light chain variable region (LCVR) comprising the amino acid sequence of SEQ ID NO: 28.

30. The method of claim 29, wherein the first antigen-binding domain comprises a HCDR1 comprising the amino acid sequence of SEQ ID NO: 17, a HCDR2 comprising the amino acid sequence of SEQ ID NO: 18, and a HCDR3 comprising the amino acid sequence of SEQ ID NO: 19.

31. The method of claim 29 or 30, wherein the first antigen-binding domain comprises a LCDR1 comprising the amino acid sequence of SEQ ID NO: 29, a LCDR2 comprising the amino acid sequence of SEQ ID NO: 30, and a LCDR3 comprising the amino acid sequence of SEQ ID NO: 31.

32. The method of any one of claims 29-31, wherein the first antigen-binding domain comprises a HCVR comprising the amino acid sequence of SEQ ID NO: 16, and a LCVR comprising the amino acid sequence of SEQ ID NO: 28.

33. The method of any one of claims 29-32, wherein the second antigen-binding domain comprises:
(a) three heavy chain complementarity determining regions (HCDR1, HCDR2 and HCDR3) contained within a heavy chain variable region (HCVR) comprising the amino acid sequence of SEQ ID NO: 20 or SEQ ID NO: 24; and
(b) three light chain complementarity determining regions (LCDR1, LCDR2 and LCDR3) contained within a light chain variable region (LCVR) comprising the amino acid sequence of SEQ ID NO: 28.

34. The method of claim 33, wherein the second antigen-binding domain comprises a HCDR1 comprising the amino acid sequence of SEQ ID NO: 21 or SEQ ID NO: 25, a HCDR2 comprising the amino acid sequence of SEQ ID NO: 22 or SEQ ID NO: 26, and a HCDR3 comprising the amino acid sequence of SEQ ID NO: 23 or SEQ ID NO: 27.

35. The method of claim 33 or 34, wherein the second antigen-binding domain comprises a LCDR1 comprising the amino acid sequence of SEQ ID NO: 29, a LCDR2 comprising the amino acid sequence of SEQ ID NO: 30, and a LCDR3 comprising the amino acid sequence of SEQ ID NO: 31.

36. The method of any one of claims 33-35, wherein the second antigen-binding domain comprises a HCVR comprising the amino acid sequence of SEQ ID NO: 20 or SEQ ID NO: 24, and a LCVR comprising the amino acid sequence of SEQ ID NO: 28.

37. The method of any one of claims 29-36, wherein the bispecific antibody comprises a human IgG heavy chain constant region.

38. The method of claim 37, wherein the human IgG heavy chain constant region is isotype IgG1.

39. The method of claim 37, wherein the human IgG heavy chain constant region is isotype IgG4.

40. The method of claim 38 or 39, wherein the bispecific antibody comprises a chimeric hinge that reduces Fcγ receptor binding relative to a wild-type hinge of the same isotype.

41. The method of any one of claims 37-40, wherein the first heavy chain or the second heavy chain, but not both, comprises a CH3 domain comprising a H435R (EU numbering) modification and a Y436F (EU numbering) modification.

42. The method of any one of claims 29-36, wherein the bispecific antibody comprises a first heavy chain comprising the amino acid sequence of SEQ ID NO: 32.

43. The method of any one of claims 29-36, wherein the bispecific antibody comprises a second heavy chain comprising the amino acid sequence of SEQ ID NO: 33.

44. The method of any one of claims 29-36, wherein the bispecific antibody comprises a second heavy chain comprising the amino acid sequence of SEQ ID NO: 34.

45. The method of any one of claims 29-36, wherein the bispecific antibody comprises a first heavy chain comprising the amino acid sequence of SEQ ID NO: 32, a second heavy chain comprising the amino acid sequence of SEQ ID NO: 33, and a common light chain comprising the amino acid sequence of SEQ ID NO: 35.

46. The method of any one of claims 29-36, wherein the bispecific antibody comprises a first heavy chain comprising the amino acid sequence of SEQ ID NO: 32, a second heavy chain comprising the amino acid sequence of SEQ ID NO: 34, and a common light chain comprising the amino acid sequence of SEQ ID NO: 35.

47. The method of any one of claims 1-46, wherein the antibody or antigen-binding fragment thereof that binds PD-1 comprises:
(a) three heavy chain complementarity determining regions (HCDR1, HCDR2 and HCDR3) contained within a heavy chain variable region (HCVR) comprising the amino acid sequence of SEQ ID NO: 36; and
(b) three light chain complementarity determining regions (LCDR1, LCDR2 and LCDR3) contained within a light chain variable region (LCVR) comprising the amino acid sequence of SEQ ID NO: 40.

48. The method of claim 47, wherein the antibody or antigen-binding fragment thereof that binds PD-1 comprises a HCDR1 comprising the amino acid sequence of SEQ ID NO: 37, a HCDR2 comprising the amino acid sequence of SEQ ID NO: 38, and a HCDR3 comprising the amino acid sequence of SEQ ID NO: 39.

49. The method of claim 47 or 48, wherein the antibody or antigen-binding fragment thereof that binds PD-1 comprises a LCDR1 comprising the amino acid sequence of SEQ ID NO:
41, a LCDR2 comprising the amino acid sequence of SEQ ID NO: 42, and a LCDR3 comprising the amino acid sequence of SEQ ID NO: 43.

50. The method of any one of claims 47-49, wherein the antibody or antigen-binding fragment thereof that binds PD-1 comprises a HCVR comprising the amino acid sequence of SEQ ID NO: 36, and a LCVR comprising the amino acid sequence of SEQ ID NO:

51. The method of claim 50, wherein the antibody or antigen-binding fragment thereof that binds PD-1 is an antibody comprising a heavy chain comprising the amino acid sequence of SEQ ID NO: 44 and a light chain comprising the amino acid sequence of SEQ ID NO: 45.

52. The method of any one of claims 1-51, wherein the bispecific antibody or antigen-binding fragment thereof is administered to the subject at a dose of from 0.03 mg to 1000 mg weekly.

53. The method of claim 52, wherein the bispecific antibody or antigen-binding fragment thereof is administered to the subject at a dose of from 0.03 mg to 900 mg weekly.

54. The method of claim 52, wherein the bispecific antibody or antigen-binding fragment thereof is administered to the subject at a dose of from 30 mg to 900 mg weekly.

55. The method of claim 52, wherein the bispecific antibody or antigen-binding fragment thereof is administered to the subject at a dose of from 100 mg to 900 mg weekly.

56. The method of claim 52, wherein the bispecific antibody or antigen-binding fragment thereof is administered to the subject at a dose of from 300 mg to 900 mg weekly.

57. The method of any one of claims 1-51, wherein the bispecific antibody or antigen-binding fragment thereof is administered to the subject at a dose of from 0.03 mg to 1000 mg once every three weeks.

58. The method of claim 57, wherein the bispecific antibody or antigen-binding fragment thereof is administered to the subject at a dose of from 0.03 mg to 900 mg once every three weeks.

59. The method of claim 57, wherein the bispecific antibody or antigen-binding fragment thereof is administered to the subject at a dose of from 30 mg to 900 mg once every three weeks.

60. The method of claim 57, wherein the bispecific antibody or antigen-binding fragment thereof is administered to the subject at a dose of from 100 mg to 900 mg once every three weeks.

61. The method of claim 57, wherein the bispecific antibody or antigen-binding fragment thereof is administered to the subject at a dose of from 300 mg to 900 mg once every three weeks.

62. The method of any one of claims 1-61, wherein the antibody or antigen-binding fragment thereof that binds PD-1 is administered to the subject at a dose of from 300 to 400 mg once every three weeks.

63. The method of claim 62, wherein the antibody or antigen-binding fragment thereof that binds PD-1 is administered to the subject at a dose of 350 mg once every three weeks.

64. The method of any one of claims 1-63, wherein the subject has stable disease, a partial response, or a complete response following administration of the bispecific antibody or antigen-binding fragment thereof for at least one week at a dose of from 0.03 mg to 900 mg in combination with the antibody or antigen-binding fragment thereof that binds PD-1.

65. The method of any one of claims 1-64, further comprising administering to the subject an IL-6R antagonist.

66. The method of claim 65, wherein the IL-6R antagonist is an anti-IL-6R antibody, optionally wherein the anti-IL-6R antibody is sarilumab or tocilizumab.

67. The method of any one of claims 1-66, wherein the subject has:
at least a 50% decline in prostate specific antigen (PSA) levels in the subject;
at least a 55% decline in PSA levels in the subject;
at least a 60% decline in PSA levels in the subject;
at least a 65% decline in PSA levels in the subject;
at least a 70% decline in PSA levels in the subject;
at least a 75% decline in PSA levels in the subject;
at least a 80% decline in PSA levels in the subject;
at least a 85% decline in PSA levels in the subject;
at least a 90% decline in PSA levels in the subject;
at least a 95% decline in PSA levels in the subject;
at least a 96% decline in PSA levels in the subject;
at least a 97% decline in PSA levels in the subject;
at least a 98% decline in PSA levels in the subject;
at least a 99% decline in PSA levels in the subject;
a reduction in the size of at least one lesion that has a PSMA PET signal less than the PSMA PET signal in the subject's liver; and/or
a response in the subject following pseudo-progression,
following administration of the bispecific anti-PSMA x anti-CD28 antibody or antigen-binding fragment thereof and the anti-PD-1 antibody or antigen-binding fragment thereof.

68. A method of treating a solid tumor in a subject in need thereof, comprising administering to the subject a combination of a bispecific antibody or antigen-binding fragment thereof comprising a first antigen-binding domain that specifically binds a tumor-associated antigen on the tumor cell, and a second antigen-binding domain that specifically binds human CD28 on a T cell, and an antibody or antigen-binding fragment thereof that specifically binds programmed death receptor-1 (PD-1).
